(19) United States
(12) Patent Application Publication
(10) Pub. No.: US 2012/0215034 A1
(43) Pub. Date: Aug. 23, 2012

(54) PROCESS FOR CONVERTING HYDROCARBON FEEDSTOCKS WITH ELECTROLYTIC AND PHOTOELECTROCATALYTIC RECOVERY OF HALOGENS

(76) Inventor: Eric McFarland, Santa Barbara, CA (US)

(21) Appl. No.: 13/458,483

(22) Filed: Apr. 27, 2012

Related U.S. Application Data
(62) Division of application No. 12/765,114, filed on Apr. 22, 2010.
(60) Provisional application No. 61/171,572, filed on Apr. 22, 2009.

(51) Int. Cl. C07C 29/58; C07C 31/04

(52) U.S. Cl. 568/893

(57) ABSTRACT

A method for converting a hydrocarbon feedstock into higher hydrocarbons is provided comprising reacting a hydrocarbon feedstock with a molecular halogen to form alkyl halides; reacting at least a portion of the alkyl halide in the presence of a catalyst to form higher hydrocarbons and a hydrogen halide; and converting at least a portion of the hydrogen halide into the molecular halogen via photoelectrocatalysis. Additional methods are also provided.

### CROSS REFERENCE TO RELATED APPLICATIONS

This application is a divisional application of U.S. patent application Ser. No. 12/765,114, filed Apr. 22, 2010, which claims priority to U.S. Patent Provisional Application Ser. No. 61/171,572, filed Apr. 22, 2009, U.S. patent application Ser. No. 12/152,515, filed May 14, 2008, and U.S. patent application Ser. No. 11/703,358, filed Feb. 5, 2007, the entire contents of which are incorporated by reference herein.

### BACKGROUND

This invention generally relates to carbon-carbon coupling and, more particularly, to methods for converting various hydrocarbon feedstocks into useful products using electrolytic and photoelectrocatalytic recovery of halogens.

Scientists have long sought efficient ways to convert methane and other hydrocarbons into longer chain hydrocarbons, olefins, aromatic hydrocarbons, and other products. C—H bond activation has been the focus of intense research for decades, with mixed results. More efficient processes could create value in a number of ways, including facilitating the utilization of hydrocarbon feedstocks (e.g., stranded natural gas, biomass sources, etc.) through conversion into more easily transportable and useful fuels and feedstocks, and allowing the use of inexpensive feedstocks (e.g., methane and other light hydrocarbons) for end products often made from higher hydrocarbons.

U.S. Pat. No. 6,525,230 discloses methods of converting alkanes to other compounds using a “zone reactor” comprised of a hollow, unsegregated interior defining first, second, and third zones. Oxygen reacts with metal bromide in the first zone to provide bromine; bromine reacts with the alkane in the second zone to form alkyl bromide and hydrogen bromide; and the alkyl bromide reacts with metal oxide in the third zone to form the corresponding product. In one embodiment, the flow of gases through the reactor may be reversed to convert the metal oxide back to metal bromide and to convert the metal bromide back to the metal oxide. The reactor may be operated in a cyclic mode.

U.S. Pat. No. 6,452,058 discloses an oxidative halogenation process for producing alkyl halides from an alkane, hydrogen halide, and, preferably, oxygen, using a rare earth halide or oxyhalide catalyst. The alternative of using molecular halogen is also mentioned. Other patents, such as U.S. Pat. Nos. 3,172,915, 3,657,367, 4,769,504, and 4,795,843, disclose the use of metal halide catalysts for oxidative halogenation of alkanes. Oxidative halogenation, however, may include the production of perhalogenated products and a quantity of deep oxidation products (CO and CO₂).

The oxychlorination process may remove the water from HCl (a costly step) and then react the HCl with oxygen and hydrocarbon directly. Oxychlorination processes rely on the separation of HCl from the unreacted alkanes and higher hydrocarbon products by using water absorption, and subsequent recovery of anhydrous HCl from the aqueous hydrochloric acid. U.S. Pat. No. 2,220,570 discloses a process and apparatus for the absorption of HCl in water where the heat of absorption is dissipated by contacting the HCl gas with ambient air, and also by the vaporization of water. A process for producing aqueous hydrochloric acid with a concentration of at least 35.5 wt % by absorbing gaseous HCl in water is disclosed in U.S. Pat. No. 4,488,884. U.S. Pat. No. 3,779,870 teaches a process for the recovery of anhydrous HCl gas by extractive distillation using a chloride salt. U.S. Pat. No. 4,259,309 teaches a method for producing gaseous HCl from dilute aqueous HCl using an amine together with an inert water-immiscible solvent.

Although researchers have made some progress in the search for more efficient CH bond activation pathways for converting natural gas and other hydrocarbon feedstocks into fuels and other products, there remains a tremendous need for a continuous, economically viable, and more efficient process.

### SUMMARY

This invention generally relates to carbon-carbon coupling and, more particularly, to methods for converting various hydrocarbon feedstocks into useful products using electrolytic and photoelectrocatalytic recovery of halogens.

The present invention combines the thermal (non-electrochemical) reactivity of halogens (preferably bromine) with hydrocarbons to produce hydrogen halide (preferably HBr) and reactive alkyl halides or other carbon-containing intermediates that may be converted to subsequent products, more readily than the original hydrocarbon, with the facile electrolysis of hydrogen halides or halide salts to create an overall process with significantly higher efficiency. The use of halogens prevents the total oxidation of the hydrocarbon to carbon dioxide and allows subsequent production of partial oxidation products.

In one aspect of the invention, a method for converting a hydrocarbon feedstock into higher hydrocarbons comprises reacting a hydrocarbon feedstock with a molecular halogen to form alkyl halides; reacting at least a portion of the alkyl halide in the presence of a catalyst to form higher hydrocarbons and a hydrogen halide; and converting at least a portion of the hydrogen halide into the molecular halogen via photoelectrocatalysis.

In another aspect of the invention, a method for converting a hydrocarbon feedstock into methanol comprises reacting a hydrocarbon feedstock with a molecular halogen to form alkyl halides; reacting at least a portion of the alkyl halide with an alkali to form methanol and a halide salt; and converting at least a portion of the halide salt into the molecular halogen via photoelectrocatalysis.

In another aspect of the invention, a method for converting a hydrocarbon feedstock into alkyl amines comprises reacting a hydrocarbon feedstock with a molecular halogen to form alkyl halides; reacting at least a portion of the alkyl halides with ammonia or an ammonium species to form alkyl amines and a halide salt; and converting at least a portion of the halide salt into the molecular halogen via photoelectrocatalysis.

In yet another aspect of the invention, a method for converting coal into coke comprises reacting coal with a molecular halogen to form halogenated coal intermediates; reacting at least a portion of the halogenated coal intermediates in the presence of a catalyst to form coke and a hydrogen halide; and converting at least a portion of the hydrogen halide into the molecular halogen via photoelectrocatalysis.

In yet another embodiment, a method for converting coal or a biomass-derived hydrocarbon feedstock into polyols comprises reacting coal or a biomass-derived hydrocarbon feedstock with a molecular halogen to form alkyl halides; reacting at least a portion of the alkyl halides with an alkali to form polyols and a halide salt; and converting at least a portion of the halide salt into the molecular halogen via photoelectrocatalysis.

The features and advantages of the present invention will be apparent to those skilled in the art. While numerous changes may be made by those skilled in the art, such changes are within the spirit of the invention.

### BRIEF DESCRIPTION OF THE DRAWINGS

FIG. 1 is a schematic diagram of a continuous process for converting a hydrocarbon feedstock into higher hydrocarbons according to one embodiment of the invention;

FIG. 2 is a schematic diagram of a continuous process for converting a hydrocarbon feedstock into higher hydrocarbons according to another embodiment of the invention;

FIG. 3 is a schematic diagram of a continuous process for converting a hydrocarbon feedstock into methanol according to one embodiment of the invention, in which a membrane-type electrolytic cell is used to regenerate molecular bromine;

FIG. 4 is a schematic diagram of a continuous process for converting a hydrocarbon feedstock into methanol according to another embodiment of the invention, in which a diaphragm-type electrolytic cell is used to generate molecular bromine;

FIG. 5 is a schematic diagram of a continuous process for converting a hydrocarbon feedstock into higher hydrocarbons in which an oxygen-depolarized cathode is provided, according to one embodiment of the invention;

FIG. 6. is a schematic illustration of an electrolytic cell according to one embodiment of the invention;

FIG. 7 is a schematic illustration of a continuous process for converting coal into coke and hydrogen, according to one embodiment of the invention;

FIG. 8 is a schematic illustration of a process for converting coal or biomass into polyols and hydrogen, according to one embodiment of the invention;

FIG. 9 is a chart illustrating product selectivity for bromination of methane according to one embodiment of the invention;

FIG. 10 is a chart illustrating product selectivity for coupling of methyl bromide according to one embodiment of the invention;

FIG. 11 is a chart illustrating product selectivity for coupling of methyl bromide according to one embodiment of the invention;

FIG. 12 is a schematic diagram of a continuous process for converting a hydrocarbon feedstock into higher hydrocarbons according to one embodiment of the invention, in which a photoelectrocatalytic cell is used to regenerate molecular bromine;

FIG. 13 is a schematic diagram of a continuous process for converting a hydrocarbon feedstock derived from a biomass source into higher hydrocarbons according to one embodiment of the invention, in which a photoelectrocatalytic cell is used to regenerate molecular bromine;

FIG. 14 is a schematic diagram of a continuous process for converting a biomass feedstock into higher hydrocarbons according to one embodiment of the invention, in which a photoelectrocatalytic cell is used to regenerate molecular bromine; and

FIG. 15 is a schematic diagram of a continuous process for converting a feedstock comprising coal into higher hydrocarbons according to one embodiment of the invention, in which a photoelectrocatalytic cell is used to regenerate molecular bromine.

### DETAILED DESCRIPTION

This invention generally relates to carbon-carbon coupling and, more particularly, to methods for converting various hydrocarbon feedstocks into useful products using electrolytic and photoelectrocatalytic recovery of halogens.

The present invention provides a chemical process for converting hydrocarbon feedstocks into higher value products, such as fuel-grade hydrocarbons, methanol, aromatics, amines, coke, and polyols, using molecular halogen to activate C—H bonds in the feedstock and electrolysis to convert hydrohalic acid (hydrogen halide) or halide salts (e.g., sodium bromide) formed in the process back into molecular halogen. Nonlimiting examples of hydrocarbon feedstocks appropriate for use in the present invention may include alkanes (e.g., methane, ethane, propane, and even larger alkanes); olefins; natural gas and other mixtures of hydrocarbons; biomass-derived hydrocarbons; and coal. Certain oil refinery processes may yield light hydrocarbon streams (so-called “light-ends”), typically a mixture of C₁-C₃ hydrocarbons, which may be used with or without added methane as the hydrocarbon feedstock. With the exception of coal, in most cases the feedstock may be primarily aliphatic in nature.

The hydrocarbon feedstock may be converted into higher products by reaction with molecular halogen, as described in more detail below. Bromine (Br₂) and chlorine (Cl₂) are preferred, with bromine being most preferred, in part because the over potential required to convert Br⁻ to Br₂ is significantly lower than that required to convert Cl⁻ to Cl₂ (1.09V for Br⁻ vs. 1.36V for Cl⁻). Fluorine and iodine may be used, though not necessarily with equivalent results. Some of the problems associated with fluorine may likely be addressed by using dilute streams of fluorine (e.g., fluorine gas carried by helium, nitrogen, or other diluent). It is expected, however, that more vigorous reaction conditions may be required for alkyl fluorides to couple and form higher hydrocarbons, due to the strength of the fluorine-carbon bond. Similarly, problems associated with iodine (e.g., the endothermic nature of certain iodine reactions) may likely be addressed by carrying out the halogenation and/or coupling reactions at higher temperatures and/or pressures. While bromine and hydrogen bromide may be used in the descriptions contained herein, it should be understood that chlorine, flourine, or iodine may be substituted for bromine in all of the processes unless otherwise specifically stated.

As used herein, the term “higher hydrocarbons” may refer to hydrocarbons having a greater number of carbon atoms than one or more components of the hydrocarbon feedstock, as well as olefinic hydrocarbons having the same or a greater number of carbon atoms as one or more components of the hydrocarbon feedstock. For instance, if the feedstock is natural gas—typically a mixture of light hydrocarbons, predominantly methane, with lesser amounts of ethane, propane and butane, and even smaller amounts of longer chain hydrocarbon such as pentane, hexane, etc.—the “higher hydrocarbon(s)” produced according to the invention may include a C₂ or higher hydrocarbon, such as ethane, propane, butane, C₅+ hydrocarbons, aromatic hydrocarbons, etc., and optionally ethylene, propylene and/or longer olefins. The term “light hydrocarbons” (sometimes abbreviated “LHCs”) may refer to C₁-C₄ hydrocarbons, e.g., methane, ethane, propane, ethylene, propylene, butanes, and butenes, all of which are normally gasses at room temperature and atmospheric pressure. Fuel grade hydrocarbons typically have 5 or more carbons and are liquids at room temperature.

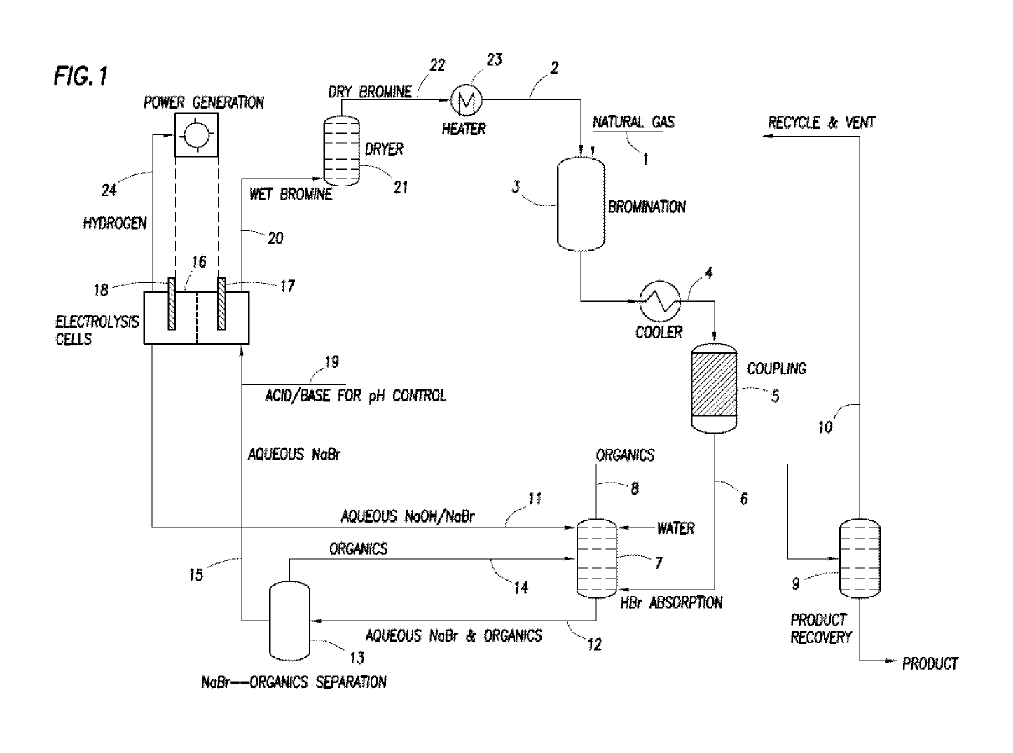

FIGS. 1-5 are schematic flow diagrams generally depicting different embodiments of the invention, in which a hydrocarbon feedstock may react with molecular halogen (e.g., bromine) and be converted into one or more higher value products. Referring to FIG. 1, one embodiment of a process for making higher hydrocarbons from natural gas, methane, or other light hydrocarbons is depicted. The feedstock (e.g., natural gas) and molecular bromine are carried by separate lines 1, 2 into a bromination reactor 3 and allowed to react. Products (e.g., HBr, alkyl bromides, optionally olefins), and possibly unreacted hydrocarbons, may exit the reactor and be carried by a line 4 into a carbon-carbon coupling reactor 5. Optionally, the alkyl bromides may be routed to a separation unit (not shown), where monobrominated hydrocarbons and HBr may be separated from polybrominated hydrocarbons, with the latter being carried back to the bromination reactor to undergo “reproportionation” with methane and/or other light hydrocarbons.

In the coupling reactor 5, monobromides and possibly other alkyl bromides and olefins may react in the presence of a coupling catalyst to form higher hydrocarbons. HBr, higher hydrocarbons, and (possibly) unreacted hydrocarbons and alkyl bromides may exit the coupling reactor and be carried by a line 6 to a hydrogen bromide absorption unit 7, where hydrocarbon products may be separated from HBr via absorption, distillation, and/or some other suitable separation technique. Hydrocarbon products may be carried away by a line 8 to a product recovery unit 9, which may separate the higher hydrocarbon products from any residual natural gas or other gaseous species, which may be vented through a line 10 or, in the case of natural gas or lower alkanes, recycled and carried back to the bromination reactor. Alternatively, combustible species may be routed to a power generation unit and may be used to generate heat and/or electricity for the system.

Aqueous sodium hydroxide or other alkali may be carried by a line 11 into the HBr absorption unit, where it may neutralize at least some of the HBr, and form aqueous sodium bromide. The aqueous sodium bromide and minor amounts of hydrocarbon products and other organic species may be carried by a line 12 to a separation unit 13, which may operate via distillation, liquid-liquid extraction, flash vaporization, or some other suitable method to separate the organic components from the sodium bromide. The organics may either be routed away from the system to a separate product cleanup unit or, in the embodiment shown, returned to the HBr absorption unit 7 through a line 14 and ultimately exit the system via line 8.

Aqueous sodium bromide may be carried from the NaBr-organics separation unit 13 by a line 15 to an electrolytic cell 16, having an anode 17, and a cathode 18. An inlet line 19 may be provided for the addition of water, additional electrolyte, and/or acid or alkali for pH control. More preferably, a series of electrolytic cells, rather than a single cell, may be used as an electrolyzer. As an alternative, several series of cells may be connected in parallel. Nonlimiting examples of electrolytic cells include diaphragm, membrane, and mercury cell, which may be mono-polar or di-polar. The exact material flows with respect to make-up water, electrolyte, and other process features may vary with the type of cell used. Aqueous sodium bromide may be electrolyzed in the electrolytic cell(s), with bromide ion being oxidized at the anode (2 Br⁻ →Br₂+2e⁻) and water being reduced at the cathode (2H₂O+2e⁻ →H₂+2OH⁻). Aqueous sodium hydroxide may be removed from the electrolyzer and routed to the HBr absorption unit via line 11.

Bromine and hydrogen produced in the electrolyzer may be recovered, with bromine being recycled and used again in the process. Specifically, wet bromine may be carried by a line 20 to a dryer 21, and dry bromine may be carried by a line 22 to a heater 23, and then by line 2 back into the bromination reactor 3. In instances where the amount of water associated with the bromine is tolerable in bromination and coupling, the dryer may be eliminated. Hydrogen produced at the anode of the electrolytic cell can be off-gassed or, more preferably, collected, compressed, and routed through a line 24 to a power generation unit, such as a fuel cell or hydrogen turbine. Alternatively, hydrogen produced may be recovered for sale or other use. Any electrical power that is generated may be used to power various pieces of equipment employed in the continuous process, including the electrolytic cells.

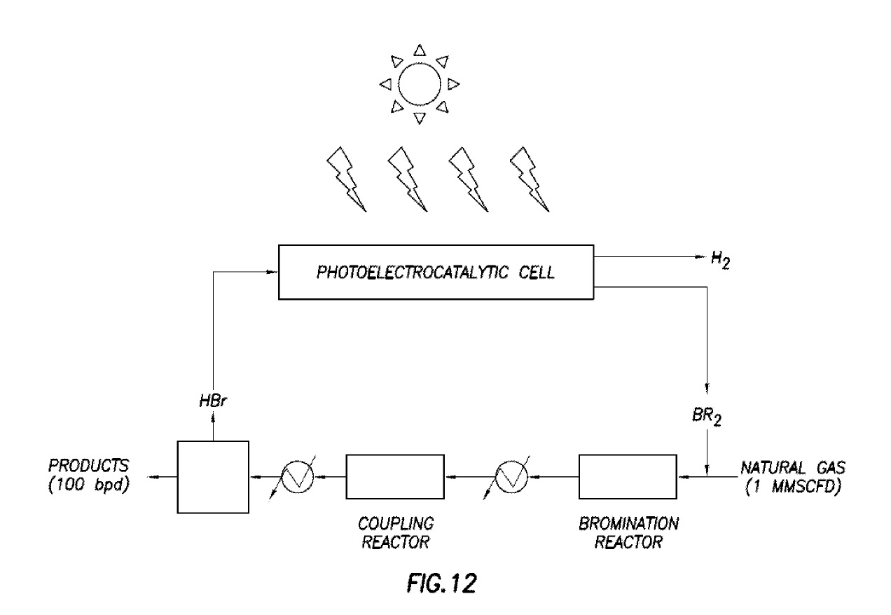

In other embodiments shown in FIGS. 12-15, a photoelectrocatalytic process may be used. In these embodiments, a process similar to that described above using an electrolyzer may be carried out using a photoelectrocatalytic cell powered by solar radiation. The photoelectrocatalytic cell may be used in the same manner as the electrolyzer to oxidize HBr to form hydrogen and molecular bromine, as described in more detail below.

Various embodiments and features of individual subprocesses and other improvements for carrying out the invention will now be described in more detail.

Bromination

Bromination of the hydrocarbon feedstock may be carried out in a fixed bed, fluidized bed, or other suitable reactor, at a temperature and pressure such that the bromination products and reactants may be gases, for example, 1-50 atm, 150-600° C., more preferably 400-600° C., even more preferably, 450-515° C., with a residence time of 1-60 seconds, more preferably 1-15 seconds. Higher temperatures tend to favor coke formation, while low temperatures require larger reactors. Using a fluidized bed, or moving bed reactor configuration may offer the advantage of improved heat transfer.

Alkane bromination may be initiated using heat or light, with thermal means being preferred. In one embodiment, the reactor may also contain a halogenation catalyst, such as a zeolite, amorphous alumino-silicate, acidic zirconia, tungstates, solid phosphoric acids, metal oxides, mixed metal oxides, metal halides, mixed metal halides (the metal in such cases being, e.g., nickel, copper, cerium, cobalt, etc.), and/or or other catalysts as described, e.g., in U.S. Pat. Nos. 3,935,289 and 4,971,664, which are incorporated herein in their entirety. In an alternate embodiment, the reactor may contain a porous or non-porous inert material that may provide sufficient surface area to retain coke formed in the reactor and prevent it from escaping. The inert material may also promote the formation of polyhalogenated hydrocarbons, such as tribromopropane. In still another embodiment, both a catalyst and an inert material may be provided in the reactor. Optionally, the reactor may contain different regions or zones to allow, in or more zones, complete conversion of molecular bromine to produce alkyl bromides and hydrogen bromide.

The bromination reaction may also be carried out in the presence of an isomerization catalyst, such as a metal bromide (e.g., NaBr, KBr, CuBr, NiBr₂, MgBr₂, CaBr₂,), metal oxide (e.g., SiO₂, ZrO₂, Al₂O₃,), or metal (Pt, Pd, Ru, Ir, Rh) to help generate the desired brominated isomer(s). Since isomerization and bromination conditions are similar, the bromination and isomerization may be carried out in the same reactor vessel. Alternatively, a separate isomerization reactor may be utilized, located downstream of the bromination reactor and upstream of the coupling reactor.

Reproportionation

In some embodiments, “reproportionation” of polyhalogenated hydrocarbons (polyhalides), i.e., halogenated hydrocarbons containing two or more halogen atoms per molecule may be carried out. Monohalogenated alkanes (monohalides) created during the halogenation reaction may be desirable as predominant reactant species for subsequent coupling reactions and formation of higher molecular weight hydrocarbons. For certain product selectivities, polyhalogenated alkanes may be desirable. Reproportionation may allow a desired enrichment of monohalides to be achieved by reacting polyhalogenated alkyl halides with nonhalogenated alkanes, generally in the substantial absence of molecular halogens, to control the ratio of mono-to-polyhalogenated species. For example, dibromomethane may be reacted with methane to produce methyl bromide; dibromomethane may be reacted with propane to produce methyl bromide and propyl bromide and/or propylene; and so forth.

Reactive reproportionation may be accomplished by allowing the hydrocarbon feedstock and/or recycled alkanes to react with polyhalogenated species from the halogenation reactor, preferably in the substantial absence of molecular halogen. As a practical matter, substantially all of the molecular halogen entering the halogenation reactor may be quickly consumed, forming mono- and polyhalides; therefore reproportionation of higher bromides may be accomplished simply by introducing polybromides into a mid- or downstream region or “zone” of the halogenation reactor, optionally heated to a temperature that differs from the temperature of the rest of the reactor.

Alternatively, reproportionation may be carried out in a separate “reproportionation reactor,” where polyhalides and unhalogenated alkanes are allowed to react, preferably in the substantial absence of molecular halogen. In this embodiment, natural gas or another hydrocarbon feedstock and molecular bromine may be carried by separate lines a heated bromination reactor and allowed to react. Products (HBr, alkyl bromides) and possibly unreacted hydrocarbons, may exit the reactor and be carried into a first separation unit (SEP I), where monobrominated hydrocarbons and HBr may be separated from polybrominated hydrocarbons. The monobromides, HBr, and possibly unreacted hydrocarbons may be carried through a heat exchanger to a coupling reactor, and allowed to react. The polybromides may be carried to a reproportionation reactor. Additional natural gas or other alkane feedstock may be introduced into the reproportionation reactor. Polybromides may react with unbrominated alkanes in the reproportionation reactor to form monobromides, which may be carried to the coupling reactor after first passing through a heat exchanger.

In another embodiment of the invention, where the hydrocarbon feedstock comprises natural gas containing a considerable amount of C₂ and higher hydrocarbons, the “fresh” natural gas feed may be introduced directly into the reproportionation reactor, and recycled methane (which passes through the reproportionation reactor unconverted) may be carried back into the halogenation reactor.

Reproportionation may be thermally driven and/or facilitated by use of a catalyst. Nonlimiting examples of suitable catalysts include metal oxides, metal halides, and zeolites. U.S. Pat. No. 4,654,449 discloses the reproportionation of polyhalogenated alkanes with alkanes using an acidic zeolite catalyst. U.S. Pat. Nos. 2,979,541 and 3,026,361 disclose the use of carbon tetrachloride as a chlorinating agent for methane, ethane, propane and their chlorinated analogues. All three patents are incorporated by reference herein in their entirety. Using reproportionation in the context of a continuous process for the enrichment of reactive feed stocks for the production of higher hydrocarbons has never been disclosed to our knowledge.

Reproportionation of C₁-C₅ alkanes with dibromomethane and/or other polybromides may occur at temperatures ranging from 350 to 550° C., with the optimal temperature depending on the polybromide(s) that are present and the alkane(s) being brominated. In addition, reproportionation may proceed more quickly at elevated pressures (e.g., 2-30 bar). By achieving a high initial methane conversion in the halogenation reactor, substantial amounts of di- and tribromomethane may be created; those species may then be used as bromination reagents in the reproportionation step. Using di- and tribromomethane allows for controlled bromination of C₁-C₅ alkanes to monobrominated C₁-C₅ bromoalkanes and C₂-C₅ olefins. Reproportionation of di- and tribromomethane facilitates high initial methane conversion during bromination, which should reduce the methane recycle flow rate and enrich the reactant gas stream with C₂-C₅ monobromoalkanes and olefins, which may couple to liquid products over a variety of catalysts, including zeolites.

In another embodiment of the invention, reproportionation may be carried out without first separating the polyhalides in a separation unit. This may be facilitated by packing the “reproportionation zone” with a catalyst, such as a zeolite, that may allow the reaction to occur at a reduced temperature. For example, although propane reacts with dibromomethane to form bromomethane and bromopropane (an example of “reproportionation”), the reaction does not occur to an appreciable degree at temperatures below about 500° C. The use of a zeolite may allow reproportionation to occur at a reduced temperature, enabling species such as methane and ethane to be brominated in one zone of the reactor, and di-, tri-, and other polybromides to be reproportionated in another zone of the reactor.

Bromine Recovery During Decoking

Inevitably, coke formation will occur in the halogenation and reproportionation processes. If catalysts are used in the reactor(s) or reactor zone(s), the catalysts may be deactivated by the coke; therefore, periodic removal of the carbonaceous deposits may be required. In addition, we have discovered that, within the coke that is formed, bromine may also be found, and it is highly desirable that this bromine be recovered in order to minimize loss of bromine in the overall process, which is important for both economic and environmental reasons.

Several forms of bromides may be present: HBr, organic bromides such as methyl bromide and dibromomethane, and molecular bromine. The invention provides means for recovering this bromine from the decoking process. In an embodiment, a given reactor may be switched off-line and air or oxygen may be introduced to combust the carbon deposits and produce HBr from the residual bromine residues. The effluent gas may be added to the air (or oxygen) reactant stream fed to the bromine generation reactor, thereby facilitating complete bromine recovery. This process may be repeated periodically.

While a given reactor is off-line, the overall process may, nevertheless, be operated without interruption by using a reserve reactor, which may be arranged in parallel with its counterpart reactor. For example, twin bromination reactors and twin coupling reactors may be utilized, with process gasses being diverted away from one, but not both, bromination reactors (or coupling reactors) when a decoking operation is desired. The use of a fluidized bed may reduce coke formation and facilitate the removal of heat and catalyst regeneration.

Another embodiment of the decoking process involves non-oxidative decoking using an alkane or mixture of alkanes, which may reduce both the loss of adsorbed products and the oxygen requirement of the process. In another embodiment of the decoking process, an oxidant such as oxygen, air, or enriched air is co-fed into the bromination section to convert the coke into carbon dioxide and/or carbon monoxide during the bromination reaction, thus eliminating or reducing the off-line decoking requirement.

Alkyl Halide Separation

The presence of large concentrations of polyhalogenated species in the feed to the coupling reactor may result in an increase in coke formation. In many applications, such as the production of aromatics and light olefins, it may be desirable to feed only monohalides to the coupling reactor to improve the conversion to products. In one embodiment of the invention, a specific separation step may be added between the halogenation/reproportionation reactor(s) and the coupling reactor.

For example, a distillation column and associated heat exchangers may be used to separate the monobromides from the polybrominated species by utilizing the large difference in boiling points of the compounds. The polybrominated species that may be recovered as the bottoms stream may be reproportionated with alkanes to form monobromide species and olefins, either in the bromination reactor or in a separate reproportionation reactor. The distillation column may be operated at any pressure of from 1 to 50 bar. The higher pressures allow higher condenser temperatures to be used, thereby reducing the refrigeration requirement.

In an embodiment, alkyl bromides from the bromination reactor may be cooled by passing through a heat exchanger, and then be passed to a distillation column equipped with two heat exchangers. At the bottom of the column, a heat exchanger may act as a reboiler, while at the top of the column the other heat exchanger may act as a partial condenser. This configuration may allow a liquid “bottoms” enriched in polybromides (and containing no more than a minor amount of monobromides) to be withdrawn from the distillation column. The polybromides may be passed through another heat exchanger to convert them back to a gas before they are returned to the bromination reactor (or sent to a separate reproportionation reactor) for reproportionation with unbrominated alkanes. At the top of the column, partial reflux of the liquid from the reflux drum may be facilitated by the upper heat exchanger, yielding a vapor enriched in lighter components including methane and HBr, and a liquid stream comprised of monobromides and HBr (and containing no more than a minor amount of polybromides).

Alternate distillation configurations may include a side stream column with and without a side stream rectifier or stripper. If the feed from the bromination reactor contains water, the bottoms stream from the distillation column may also contain water, and a liquid-liquid phase split on the bottoms stream may be used to separate water from the polybrominated species. Due to the presence of HBr in the water stream, it may either be sent to a HBr absorption column or to the bromine generation reactor.

Catalytic Coupling of Alkyl Halides to Higher Molecular Weight Products

The alkyl halides produced in the halogenation/reproportionation step may be reacted over a catalyst to produce higher hydrocarbons and hydrogen halide. The reactant feed may also contain hydrogen halide and unhalogenated alkanes from the bromination reactor. According to the invention, any of a number of catalysts may be used to facilitate the formation of higher hydrocarbon products from halogenated hydrocarbons. Nonlimiting examples may include non-crystalline alumino silicates (amorphous solid acids), tungsten/zirconia super acids, sulfated zirconia, alumino phosphates such as SAPO-34 and its framework-substituted analogues (substituted with, e.g., Ni or Mn), Zeolites, such as ZSM-5 and its ion-exchanged analogs, and framework substituted ZSM-5 (substituted with Ti, Fe, Ti+Fe, B, or Ga). Preferred catalysts for producing liquid-at-room-temperature hydrocarbons include ion-exchanged ZSM-5 having a SiO₂/Al₂O₃ ratio below 300, preferably below 100, and most preferably 30 or below. Nonlimiting examples of preferred exchanged ions include ions of Ag, Ba, Bi, Ca, Fe, Li, Mg, Sr, K, Na, Rb, Mn, Co, Ni, Cu, Ru, Pb, Pd, Pt, and Ce. These ions can be exchanged as pure salts or as mixtures of salts. The preparation of doped zeolites and their use as carbon-carbon coupling catalysts is described in Patent Publication No. US 2005/0171393 A1, which is incorporated by reference herein in its entirety.

In one embodiment of the invention a Mn-exchanged ZSM-5 zeolite having a SiO₂/Al₂O₃ ratio of 30 may be used as the coupling catalyst. Under certain process conditions, it may produce a tailored selectivity of liquid hydrocarbon products.

Coupling of haloalkanes may be carried out in a fixed bed, fluidized bed, or other suitable reactor, at a suitable temperature (e.g., 150-600° C., preferably 275-425° C.) and pressure (e.g., 0.1 to 35 atm) and a residence time (τ) of from 1-45 seconds. In general, a relatively long residence time may favor conversion of reactants to products, as well as product selectivity, while a short residence time may mean higher throughput and (possibly) improved economics. It may be possible to direct product selectivity by changing the catalyst, altering the reaction temperature, and/or altering the residence time in the reactor. For example, at a moderate residence time of 10 seconds and a moderate temperature of 350° C., xylene and mesitylenes are the predominant components of the aromatic fraction (benzene+toluene+xylenes+mesitylenes; “BTXM”) produced when the product of a methane bromination reaction is fed into a coupling reactor packed with a metal-ion-impregnated ZSM-5 catalyst, where the impregnation metal is Ag, Ba, Bi, Ca, Co, Cu, Fe, La, Li, Mg, Mn, Ni, Pb, Pd, or Sr, and the ZSM-5 catalyst is Zeolyst CBV 58, 2314, 3024, 5524, or 8014, (available from Zeolyst International (Valley Forge, Pa.)). At a reaction temperature of 425° C. and a residence time of 40 seconds, toluene and benzene are the predominant products of the BTXM fraction. Product selectivity may also be varied by controlling the concentration of dibromomethane produced or fed into the coupling reactor. Removal of reaction heat and continuous decoking and catalyst regeneration using a fluidized bed reactor configuration for the coupling reactor is anticipated in some facilities.

In one embodiment, the coupling reaction may be carried out in a pair of coupling reactors, arranged in parallel. This may allows the overall process to be run continuously, without interruption, even if one of the coupling reactors is taken off line for decoking or for some other reason. Similar redundancies may be utilized in the bromination, product separation, halogen generation, and other units used in the overall process.

Hydrocarbon Product Separation and Halogen Recovery

The coupling products may include higher hydrocarbons and HBr. In an embodiment, products that exit the coupling reactor may be cooled in a heat exchanger before being sent to an absorption column. HBr may be absorbed in water using a packed column or other contacting device. Input water and the product stream may be contacted either in a co-current or counter-current flow, with the counter-current flow preferred for its improved efficiency. HBr absorption may be carried out either substantially adiabatically or substantially isothermally. In one embodiment, the concentration of hydrobromic acid after absorption ranges from about 5 to 70 wt %, with a preferred range of about 20 to 50 wt %. The operating pressure may be 1 to 50 bar, more preferably 1 to 30 bar. In the laboratory, a glass column or glass-lined column with ceramic or glass packing may be used. In a pilot or commercial plant, one or more durable, corrosion-resistant materials (described below) may be utilized.

In one embodiment of the invention, the hydrocarbon products may be recovered as a liquid from the HBr absorption column. This liquid hydrocarbon stream may be phase-separated from the aqueous HBr stream using a liquid-liquid splitter and may be sent to the product cleanup unit. In another embodiment, the hydrocarbon products may be recovered from the HBr column as a gas stream, together with the unconverted methane and other light gases. The products may then be separated and recovered from the methane and light gases using any of a number of techniques. Nonlimiting examples include distillation, pressure swing adsorption, and membrane separation technologies.

In some embodiments, the product clean-up unit may comprise a reactor for converting halogenated hydrocarbons present in the product stream into unhalogenated hydrocarbons. For example, under certain conditions, small amounts of C₁-C₄ bromoalkanes, bromobenzene, and/or other brominated species may be formed and pass from the coupling reactor to the liquid-liquid splitter 16 and then to the product clean-up unit 17. These brominated species may be “hydrodehalogenated” in a suitable reactor. In one embodiment, such a reactor may comprise a continuous fixed bed, catalytic converter packed with a supported metal or metal oxide catalyst. Nonlimiting examples of the active component may include copper, copper oxide, palladium, and platinum, with palladium being preferred. Nonlimiting examples of support materials may include active carbon, alumina, silica, and zeolites, with alumina being preferred. The reactor may be operated at a pressure of 0-150 psi, preferably 0-5 psi, and a temperature of 250-400° C., preferably 300-350° C., with a GHSV of 1200-60 hr⁻¹, preferably about 240 hr⁻¹. When bromobenzene is passed over such a reactor, it is converted to benzene and HBr, with some light hydrocarbons (e.g., C₃-C₇) produced as byproducts. Although carbon deposition (coking) may deactivate the catalyst, the catalyst may be regenerated by exposure to oxygen and then hydrogen at, e.g., 500° C. and 400° C., respectively.

After HBr is separated from the hydrocarbon products, the unconverted methane may leave with the light gases in the vapor outlet of the HBr absorption unit. In one embodiment of the invention, unconverted methane may be separated from the light gases in a separation unit, which may operate using pressure or temperature swing adsorption, membrane-based separation, cryogenic distillation (preferable for large-scale production), or some other suitable separation process. Low methane conversions in the bromination reactor may result in the coupling products being carried with the light gases, which in turn may necessitate the recovery of these species from the lights gases. Separation technologies that may be employed for this purpose include, but are not limited to, distillation, pressure or temperature swing adsorption, and membrane-based technologies.

In another aspect of the invention, a process for separating anhydrous HBr from an aqueous solution of HBr is provided. HBr forms a high-boiling azeotrope with water; therefore, separation of HBr from the aqueous solution requires either breaking the azeotrope using an extractive agent or bypassing the azeotrope using pressure swing distillation. In an embodiment, water may be extracted in a distillation column and HBr may be obtained as the distillate stream. The distillate stream may also contain small amounts of water. In one embodiment, the distillation column may be a tray-tower or a packed column. Conventional ceramic packing may be preferred over structured packing Aqueous bromide salt, such as CaBr₂, may be added at the top of the distillation column, resulting in the extraction of water from aqueous HBr. A condenser may not be required for the column. A reboiler may be used to maintain the vapor flow in the distillation column. The diluted stream of aqueous CaBr₂ may be sent to the evaporation section, which optionally has a trayed or packed section. The bottoms stream from the column may be heated before entering the evaporation section. The bottoms stream may comprise mostly water (and no more than traces of HBr) and may leave the evaporation section.

In an embodiment, HBr may be displaced as a gas from its aqueous solution in the presence of an electrolyte that shares a common ion (Br or H⁺) or an ion (e.g. Ca²⁺ or SO₄²⁻) that has a higher hydration energy than HBr. The presence of the electrolyte pushes the equilibrium HBr_{aq} [symbol] HBr_{gas} towards gas evolution, which is further facilitated by heating the solution.

Aqueous solutions of metal bromides such as CaBr₂, MgBr₂ also KBr, NaBr, LiBr, RbBr, CsBr, SrBr₂, BaBr₂, MnBr₂, FeBr₂, FeBr₃, CoBr₂, NiBr₂, CuBr₂, ZnBr₂, CdBr₂, AlBr₃, LaBr₃, YBr₃, and BiBr₃ may be used as extractive agents, with aqueous solutions of CaBr₂, MgBr₂, KBr, NaBr, LiBr or mixtures thereof being preferred. The bottoms stream of the distillation column contains a diluted solution of the extracting agent. This stream may be sent to another distillation column or a vaporizer where water may be evaporated and the extracting agent may be concentrated before sending it back to the extractive distillation column. Sulfuric acid may be used as an extracting agent if its reaction with HBr to form bromine and sulfur dioxide can be minimized.

In another aspect of the invention, various approaches to product clean-up (separation and/or purification) are provided. A number of bromide species may be present in the unpurified product stream: HBr, organic bromides such as methyl bromide and dibromomethane, and bromo-aromatics. In one embodiment of the invention, hydrocarbon products may be separated from brominated species by passing the product stream over copper metal, NiO, CaO, ZnO, MgO, BaO, or combinations thereof. Preferably, the products may be run over one or more of the above-listed materials at a temperature of from 250-600° C., more preferably, 400-500° C. This process may generally be tolerant of any CO₂ that may be present.

In another embodiment, particularly for large-scale production of hydrocarbons, unconverted methane may be separated from other light hydrocarbons as well as heavier products (e.g., benzene, toluene, etc.) using distillation. For example, methane and other light hydrocarbons that may exit the absorption column through a gas outlet are directed to a separation unit. Any unconverted methyl bromide may be removed with the light gases and may be recycled back to the bromination/reproportionation reactor. Heavier hydrocarbons may be removed as a liquid distillate.

Electrolytic Molecular Halogen Generation

Electrolysis of aqueous solutions to produce hydrogen and oxygen may produce hydrogen using electrical energy. For example, hydrogen may be produced by dissociation of water:

H₂O→½O₂+H₂ ΔH=286 kJ/mol H₂

Although energetically unfavorable, the reaction may be driven by electrolysis using 2×10⁵ Coulombs per gram-mole H₂. Water is the source of both the hydrogen and the oxygen, and the high activation energy for oxygen production requires over potentials of approximately 1.6 Volts and a stoichiometric current. In some embodiments, the electrical energy required may be approximately 300 kJ/mol H₂.

Similarly, halogens may also be produced by electrolysis of halide brines or metal halide vapor. In halogen production by electrolysis of halide salts, e.g. the chloralkali process, halogen (Cl₂) and alkali base (NaOH) are produced from the haloanion and water in an aqueous solution of salt (NaCl). Water is again the source of the hydrogen. Similarly, bromine may be produced from bromine salts (NaBr). In the latter instance, the production of molecular halogen from the haloanion may be energetically and kinetically advantageous compared to oxygen production, requiring a lower over potential (1.1 V versus 1.6V):

H₂O+NaBr→Br₂+H₂+NaOH

With 2×10⁵ Coulombs per gram-mole H₂, the required electrical energy is reduced significantly compared to H₂O alone to approximately 200 kJ/g mol H₂.

In an embodiment, an electrolytic cell may have an anode and a cathode. An electrolyte and optionally water may be added to the electrolytic cell. In some embodiments, a series of electrolytic cells, rather than a single cell, may be used as an electrolyzer. In another embodiment, several series of cells may be connected in parallel. Nonlimiting examples of electrolytic cells may include diaphragm, membrane, and mercury cells, which can be mono-polar or di-polar. In some embodiments, an air depolarized cathode may be used. The material flows with respect to make-up water, electrolyte, and other process features will vary with the type of cell used. Anodes, cathodes, electrolytes, and other features of the electrolytic cell(s) may be selected based on a number of factors understood by the skilled person, such as throughput, current power levels, and the chemistry of the electrolysis reaction(s). Nonlimiting examples are found in U.S. Pat. Nos. 4,110,180 (Nidola et al.) and 6,368,490 (Gestermann), which is incorporated by reference herein in their entirety. Additional examples may also be found in Y. Shimizu, N. Miura, N. Yamazoe, Gas-Phase Electrolysis of Hydrocarbonic Acid Using PTFE-Bonded Electrode, Int. J. Hydrogen Energy, Vol. 13, No. 6, 345-349 (1988); D. van Velzen, H. Langenkamp, A. Moryoussef, P. Millington, HBr Electrolysis in the Ispara Mark 13A Flue Gas Desulphurization Process Electrolysis in a DEM Cell, J. Applied Electrochemistry, Vol. 20, 60-68 (1990); and S. Motupally, D. Mak, F. Freire, J. Weidner, Recycling Chlorine from Hydrogen Chloride, The Electrochemical Society Interface, Fall 1998, 32-36.

Photoelectrocatalytic Recovery of Halogens

Photoelectrocatalysis may refer to a process whereby light absorbed in semiconductor electrodes of an electrochemical cell may generate electron-hole pairs which may be separated and injected into the electrolyte at the cathode and anode, respectively, to produce reduction and oxidation reactions. A photoelectrocatalytic cell may refer to an electrolytic cell capable of carrying out photoelectrocatalysis. Hence, photoelectrocatalysis may be achieved in two steps: (1) the electrons (and holes) may first be created by photoexcitation of a semiconductor electrode in a photoelectrocatalytic cell, and (2) the electrons (and holes) may drive chemical reactions in the photoelectrocatalytic cell.

In semiconductors and insulators, electrons are confined to a number of bands of energy. The term “band gap” may refer to the energy difference between the top of the valence band and the bottom of the conduction band. Electrons may be able to jump from one band to another. However, in order for an electron to jump from a valence band to a conduction band, it requires a specific minimum amount of energy for the transition. The required energy differs with different materials. Electrons may gain enough energy to jump to the conduction band by absorbing either a phonon (heat) or a photon (light). The absorption of a photon above the band gap results in the release of an electron that may be capable of taking place in a chemical reaction, such as an electrolysis reaction.

In an embodiment, a photoelectrocatalytic cell may utilize the absorption of photons above the band gap to electrolyze various chemical compounds. The conventional photoelectrocatalytic cell may be visualized as an p-n type cell or semiconductor-metal junction which may have an electrolyte and a pair of electrodes in electrical communication with the electrolyte. As a result an n-electrolyte/p configuration (p-n type cell) may be formed having an electrolyte, an n-electrode and a p-electrode. In an p-n type cell, absorption of band gap energy (e.g., solar energy from sunlight) in the anodic n-electrode results in electron-hole pairs which may separate in the space charge layer at the surface of the electrode. Hole injection may proceed into the electrolyte, while the electron may move into the bulk of the n-electrode and around the external circuit to the cathodic p-electrode (counter-electrode). An analogous process may occur when band gap energy is absorbed in the p-electrode.

In an n-electrolyte/p semiconductor configuration, the sum of the band gaps of the two electrodes must be equal to or greater than the minimum energy required to drive a desired reaction. Simultaneous illumination by sunlight, i.e., solar radiation, of n- and p-electrodes having smaller band gaps may increase the conversion efficiency of the solar radiation, since smaller band gap semiconductors absorb more light and hence provide higher conversion efficiency. The specific available photon energy depends upon the details of the p-n type cell configuration and the semiconductor electrode properties.

Using the electrolysis of water as an example, hydroxyl anions (OH⁻) may combine with holes (h⁺) to produce oxygen and water at the n-electrode:

2h′+2OH⁻→½O₂+H₂O

At the p− or metal electrode, protons (H⁺) combine with electrons (e−) to produce hydrogen:

2e⁻+2H⁺→H₂

in an aqueous electrolyte, these two half reactions may occur at the same time.

In the processes disclosed herein, alternative example reactions capable of occurring in a photoelectrocatalytic cell may include the oxidation of a hydrogen halide to elemental halogen and the conversion of carbon dioxide or carbon monoxide to methanol or formic acid. The conversion of hydrogen halide to elemental halogen may be demonstrated by the following equations which use hydrogen bromide as an example:

Bromide anions (Br⁻) combine with holes (h⁺) to produce elemental bromine at the n-electrode:

2h⁺+2Br⁻→Br2

At the p-electrode, protons (H⁺) combine with electrons (e) to produce hydrogen:

2e⁻+2H⁺→H₂

These half reactions combine to form an overall equation:

2HBr→Br₂+H₂

As another example, carbon dioxide or carbon monoxide may be reduced to form methanol or formic acid. In this example, bromide anions (Br⁻) combine with holes (h⁺) to produce elemental bromine at the n-electrode:

2h⁺+2Br⁻→Br₂

At the p-electrode, carbon dioxide and protons (H⁺) may combine with electrons (e⁻) to produce water and methanol:

CO₂+6e⁻+6H⁺→CH₃OH+H₂O

The overall reaction may be generalized and represented as:

CO₂ (or CO)+HBr→CₙHₘOₓ+Br₂+zH₂O

In an embodiment, a photoelectrocatalytic cell that may be driven by solar energy may comprise an anodic electrode comprising at least one n-type semiconducting layer, a cathodic counter-electrode comprising at least one p-type semiconducting layer, and an electrolyte in contact with the exposed surfaces of the n- and p-electrodes. The n-type layer, the p-type layer, or both may be disposed on a supporting conductive substrate and may be electrically coupled to the substrate. The n- and p-type layers may be adjacent to each other. They may be in physical contact, or they may be separated by a small insulating section. The conductive, supporting substrates may be opaque, comprising, for example, metal foils or sheets. In an embodiment, the relative areas of the n- and p-electrodes may be adjusted such that substantially equal photon absorption rates are obtained. The size of the electrodes may depend on the band gap of each semiconducting electrode and the resulting absorption characteristics (number of photons absorbed per cm²), allowing for determination of the relative areas of the electrodes required such that the photon absorption rates (number of photons per sec) of the two electrodes may be substantially equal.

A variety of photoelectrocatalytic cell configurations may be used. For example, the cells may comprise one or more flat sheet-like structures or be arranged in one or more concentric tubular configurations. In these embodiments, the electrolyte may pass over the cell if one layer is used or may pass between adjacent cell layers in an embodiment with a plurality of cell layers. In another embodiment, a particulate form of photoelectrocatalytic cells may be used. In this embodiment, individual cells as described above may comprise small particles that form a slurry with the electrolyte. Such particles may be filtered out of the electrolyte prior to the electrolyte being removed from the photoelectrocatalytic cell, or the particles may be removed in a subsequent step and reinjected with fresh electrolyte in a cyclic flow type process. In this embodiment, the individual photoelectrolyte cells may be less than 1 cm in diameter, and may comprise any suitable shape (e.g., discs, rods, spheres, cubes, etc.).

The evolved gases generated in a photoelectrocatalytic cell may be comingled and separated in a subsequent process or an impermeable membrane may be used to prevent mixing of the gases within the cell while permitting transport of electrolyte. Phase separation may then be used to separate the evolved gas from the remaining electrolyte.

In order to increase the amount of light available for the production of holes and electrons, a solar concentrator may be used. Examples of solar concentrators include, but are not limited to, conventional reflectors, such as a parabolic or a flat mirror.

The selection of electrode materials may be constrained by two considerations: (a) the minimum band gap necessary to drive a desired reaction (e.g., the oxidation of hydrogen halide) and (b) the maximum energy available from the sun. In an embodiment, the electrode materials may also be stable electrochemically and inert to any reactions in solution.

Nonlimiting examples of suitable anode materials may include doped n-type semiconductors such as TiO₂, In₂O₃, SnO₂, GaAs, GaP, WO₃, SiC, Fe₂O₃, CdS, CuInS₂, Si, and the titanates MTiO₃, where M is at least one element of barium, strontium, the rare earth elements (atomic number 57 to 71) and the transition metal elements (Groups IB through VIIB and VIII of the Periodic Table). Examples of rare earth and transition metal titanates may include without limitation LaTiO₃ and NiTiO₃, respectively. Combinations of these materials may also be used. For example, graded band gaps or multiple heterojunction semiconductor layers may be utilized. Furthermore, such combinations may permit use of semiconductors which by themselves may not be chemically inert with respect to the electrolyte. In some embodiments, this may be accomplished by overcoating the chemically sensitive semiconductor, such as CdS or GaP, with an inert semiconductor layer, such as TiO₂, to protect the chemically sensitive semiconductor from chemical attack by the electrolyte.

Nonlimiting examples of suitable cathode materials may include doped p-type semiconductors such as GaP, GaAs, Si, Cu₂S, Cu₂O, InP, ZnSe, CdTe and CuInS₂. Combinations of these materials may also be used, e.g., graded band gaps or multiple heterojunction semiconductor layers. Furthermore, in the same manner as above, such combinations may permit use of semiconductors, which by themselves may not be chemically inert with respect to the electrolyte, by overcoating the chemically sensitive semiconductor with an inert semiconductor layer to protect the chemically sensitive semiconductor from chemical attack by the electrolyte.

In another embodiment, the photoelectrocatalytic cell may have a design that also utilizes thermal decomposition to oxidize hydrogen halides. In this embodiment, solar radiation or another heat source is used to provide heat to the electrolyte solution to provide a portion of the energy required to produce molecular hydrogen and molecular halide from a hydrogen halide solution. In some embodiments, the portion of the hydrogen halide reacting due to thermal effects may range from about 5% to about 99%. In some embodiments, thermal decomposition may be the only source of energy to drive the reaction. For example, there may be times when the available solar radiation is insufficient to drive the photoelectrocatalytic reaction but may be sufficient to drive a thermal decomposition reaction.

Recovery and Recycle of Molecular Halogen

In some embodiments, halogen generation may produce both water and molecular halogen. For example, the operating conditions of the electrolytic cell or photoelectrocatalytic cell may result in the formation of water vapor that may leave with the generated gases. Water may be separated from halogen and removed before the halogen is reacted with the hydrocarbon feedstock. Where the halogen is bromine, a bromine-water, liquid-liquid phase split may be achieved upon condensation of a mixture of these species. For example, in one embodiment of the invention, a liquid-liquid flash unit may be used to separate most of the bromine from water, simply and inexpensively. The bromine phase typically contains a very small amount of water, and may be sent directly to the bromination reactor. The water phase, however, contains 1-3 wt % bromine. However, if air is used in the bromine generation step, nitrogen and unconverted oxygen may be present with the bromine and water stream that enters the flash.

The gas leaving the flash unit may consist primarily of nitrogen and unconverted oxygen, but may carry with it some bromine and water. The amount of bromine leaving with the vapor phase depends on the temperature and pressure of the flash. The flash may be operated at temperatures ranging from 0 to 50° C.; however, a lower temperature (2 to 10° C.) is preferred to reduce bromine leaving in the vapor stream. The vapor stream may be sent to the bromine scavenging section for bromine recovery. In one embodiment, the operating pressure may range from about 1 to about 50 bar, more preferably about 1 to about 30 bar. Since water freezes at 0° C., it is not possible to substantially reduce the temperature of the flash. However, the vapor stream from the flash may be contacted with a chilled brine solution, at temperatures from about −30° C. to about 10° C. Chilled brine temperatures lower than that of the flash may substantially reduce the bromine scavenging requirement of the scavenging unit. Vaporizing the bromine by heating the brine may then occur, with further heating employed to facilitate concentration of the brine for re-use. This approach to bromine recovery may be carried out either continuously or in batch mode.

Bromine contained in the water-rich phase leaving the liquid-liquid flash may be effectively recovered by distillation. Other means, such as using an inert gas to strip the bromine from the water phase and adsorption-based methods, may not be very effective, and potentially may result in a significant loss of bromine. The presently described distillation subprocess produces bromine or bromine-water azeotrope as a distillate, which may be recycled back to the flash unit. Water may be contained in the bottoms stream. Bromine may react reversibly with water to form small amounts of HBr and HOBr. In the distillation scheme, therefore, ppm levels of HBr (and/or HOBr) may be present in the bottoms stream. A side-stream rectifier or stripper may be utilized to reduce the bromine content of the bottoms stream to produce a pure water stream. Other alternatives that may reduce the bromine content of the water to below 10 ppm range include, but are not limited to, the addition of acids such as sulfuric acid, hydrochloric acid, and phosphoric acid, in very small quantities to reduce the pH of the water stream. Lowering the pH drives the HBr and HOBr stream back to bromine and water, thereby substantially reducing the loss of bromine in the water stream. HBr present in the water stream may also be recovered using ion-exchange resins or electrochemical means.

Recovery of all Halogen for Reuse

For both economic and environmental reasons, it is preferred to minimize, if not completely eliminate, loss of halogen utilized in the overall process. Molecular bromine may have the potential to leave with vented nitrogen and unconverted oxygen if it is not captured after Br₂ generation. Bromine scavenging can be carried out in a bed containing solid CuBr or MnBr₂, either loaded on a support or used in powder form, to capture Br₂ from a gas stream that may also contain H₂O, CO₂, O₂, methane &/or N₂. In one embodiment of the invention, bromine scavenging may be performed within a range of temperatures, e.g., from about −10° C. to about 200° C. When bromine scavenging is complete, molecular bromine may be released from the bed by raising the temperature of the bed to about 220° C. or higher, preferably above about 275° C. It is important that there be little if any O₂ in the bed during bromine release, as O₂ may oxidize the metal and, over time, reduce the bromine-scavenging capacity of the bed.

Construction of Critical Process Elements with Unique Corrosion-Resistant Materials

Corrosion induced by any halogen-containing process, whether in the condensed phase or the vapor phase, may present a significant challenge in the selection of durable materials for the construction of reactors, piping, and ancillary equipment. Ceramics, such as alumina, zirconia, and silicon carbides, offer exceptional corrosion resistance to most conditions encountered in the process described herein. However, ceramics suffer from a number of disadvantages, including lack of structural strength under tensile strain, difficulty in completely containing gas phase reactions (due to diffusion or mass transport along jointing surfaces), and possibly undesirable thermal transport characteristics inherent to most ceramic materials. Constructing durable, gas-tight, and corrosion resistant process control equipment (e.g., shell and tube type heat-exchangers, valves, pumps, etc.), for operation at elevated temperatures and pressures, and over extended periods of time, may require the use of formable metals such as Au, Co, Cr, Fe, Nb, Ni, Pt, Ta, Ti, and/or Zr, or alloys of these base metals containing elements such as Al, B, C, Co, Cr, Cu, Fe, H, Ha, La, Mn, Mo, N, Nb, Ni, 0, P, Pd, S, Si, Sn, Ta, Ti, V, W, Y, and/or Zr.

Based on the individual process descriptions presented above, various embodiments of overall processes for carrying out the invention will now be described in more detail.

In one embodiment of the invention shown in FIG. 1, methane may be introduced into a plug flow reactor made of the alloy ALCOR, at a rate of 1 mole/second, and molecular bromine is introduced at a rate of 0.50 moles/second with a total residence time of a 60 seconds at about 425° C. The major hydrocarbon products include methyl bromide (85%) and dibromomethane (14%), and 0.50 moles/s of HBr is produced. The methane conversion is 46%. The products are carried by a line 4 into a coupling reactor 5, which is a packed bed reactor containing a transition metal (e.g., Mn) ion-exchanged alumina-supported ZSM5 zeolite coupling catalyst at about 425° C. In the coupling reactor 5, a distribution of higher hydrocarbons is formed, as determined by the space time of the reactor. In this example, 10 seconds is preferred to produce products that are in the gasoline range. HBr, higher hydrocarbons, and (trace) unreacted alkyl bromides exit the coupling reactor and are carried by a line 6 to a hydrogen bromide separation unit 7, where HBr is partially separated by distillation. Aqueous sodium hydroxide is introduced and allowed to react at about 150° C., forming sodium bromide and alcohols from the HBr and unreacted alkyl bromides. The aqueous and organic species are carried by a line 12 to a separation unit 13, which operates via distillation to separate the organic components from the sodium bromide. Aqueous sodium bromide is carried from the NaBr-organics separation unit 13 by line 15 to an electrolytic cell 16, having an anode 17, and a cathode 18. An inlet line 19 is provided for the addition of water, additional electrolyte, and the pH adjusted to be less then 2 by addition of acid. Electrolysis is performed in a membrane cell type. Aqueous sodium bromide is electrolyzed in the electrolytic cell, with bromide ion being oxidized at the anode (2Br⁻→Br₂+2e⁻) and water being reduced at the cathode (2H₂O+2e⁻→H₂+2OH⁻). Aqueous sodium hydroxide is removed from the electrolyzer and routed to the HBr absorption unit via line 11. Bromine and hydrogen are produced in the electrolyzer.

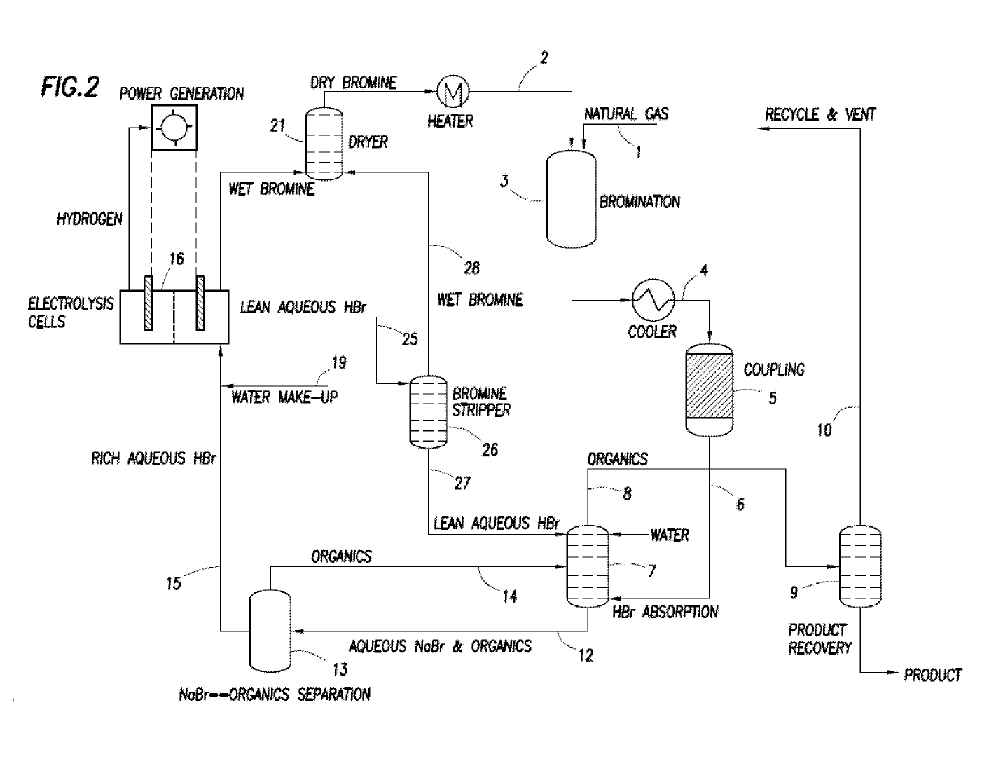

Referring to FIG. 2, an alternate embodiment for converting natural gas, methane, or other hydrocarbon feedstocks into higher hydrocarbons, such as fuel grade hydrocarbons and aromatic compounds, is depicted. In this embodiment, electrolysis takes place in a non-alkaline medium. Products from the coupling reactor (e.g., higher hydrocarbons and HBr) are carried by a line 6 to an HBr absorption unit 7, where hydrocarbon products are separated from HBr. After residual organic components are removed from the HBr in a separation unit 13, rich aqueous HBr is carried by a line 15 to the electrolytic cell 16. Make-up water, electrolyte, or acid/base for pH control, if needed, is provided by a line 19. The aqueous HBr is electrolyzed, forming molecular bromine and hydrogen. As Br₂ is evolved and removed from the electrolyzer, the concentration of HBr in the electrolyzer drops. The resulting lean aqueous HBr, along with some bromine (Br₂) entrained or dissolved therein, is carried by a line 25 to a bromine stripper 26, which separates bromine (Br₂) from lean aqueous HBr via distillation or some other suitable separation operation. The lean aqueous HBr is carried back to the HBr absorption unit by a line 27. Wet bromine is carried by a line 28 to the dryer 21, where it is dried.

In another embodiment of this aspect of the invention (not shown), natural gas, methane, or another hydrocarbon feedstock is converted into higher hydrocarbons, and halogen (e.g., Br₂) is recovered by gas phase electrolysis of hydrogen halide (e.g., HBr). Products from the coupling reactor (e.g., higher hydrocarbons and HBr) are carried by a line to an HBr absorption unit, where hydrocarbon products are separated from HBr. After residual organic components are removed from the HBr in a separation unit, gaseous HBr is carried by a line to the electrolytic cell. The gaseous HBr is electrolyzed, forming molecular bromine and hydrogen. Wet bromine is carried by a line to the dryer, where it is dried. Optionally, if dry HBr is fed to the electrolysis cells, the dryer can be eliminated.

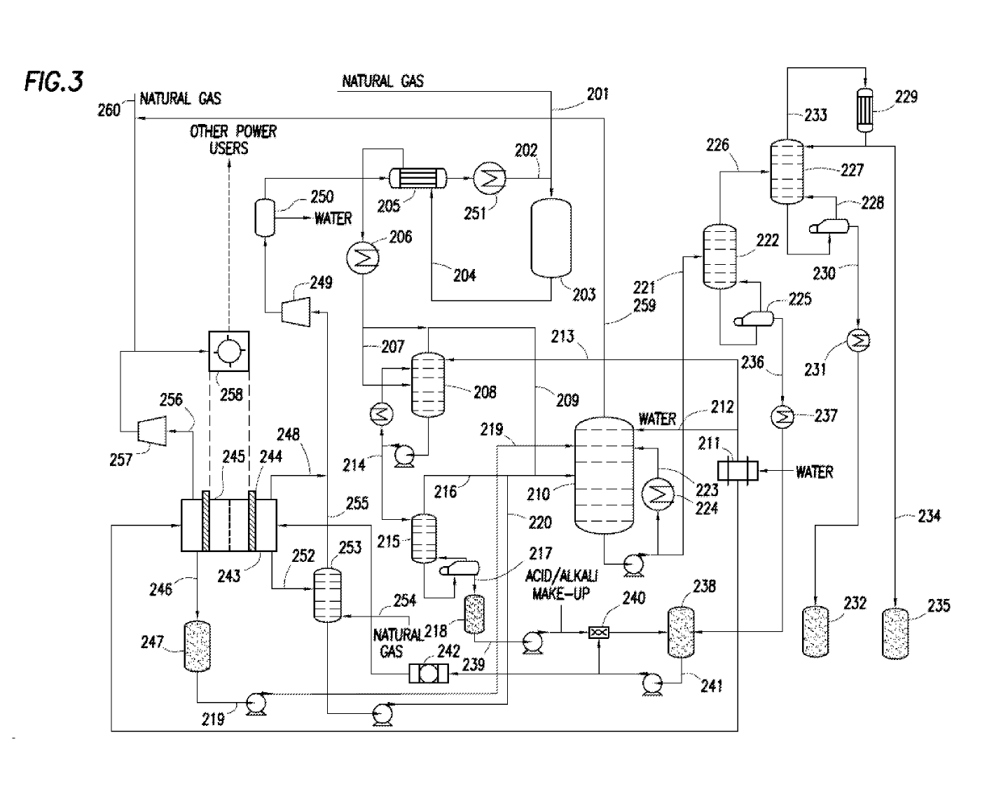

FIG. 3 depicts one embodiment of another aspect of the invention, in which natural gas, methane, or another hydrocarbon feedstock is converted into methanol via the intermediate, methyl bromide. Natural gas and gaseous bromine are carried by separate lines 201 and 202 into a bromination reactor 203 and allowed to react. The products (e.g., methyl bromide and HBr), and possibly unreacted hydrocarbons, are carried by a line 204 through a heat exchanger 205, which lowers their temperature. If necessary, the gasses are further cooled by passing through a cooler 206. A portion of the gasses 206 are carried by a line 207 to an HBr absorber 208. The remainder by-passes the HBr absorber and are carried by a line 209 directly to the reactor/absorber 210. The split proportions are determined by the acid/base disproportionation needed to achieve the proper pH in the reactor absorber.

Water, optionally pre-treated in, e.g., a reverse osmosis unit 211 to minimize salt content, is provided to the methanol reactor 210 via line 212. In addition, a separate line 213 carries water to the HBr absorber 208.

HBr solution formed in the HBr absorber 208 is sent via a line 214 to a stripper 215 where organics are separated by stripping or other means and then sent to the reactor/absorber 210 via a line 216. Gasses from the HBr absorber join the by-passed stream from the cooler 206 and are carried by a line 209 to the reactor/absorber 210. HBr solution from the stripper 215 is carried by a line 217 to an HBr holding tank 218.

Aqueous sodium hydroxide (e.g., 5-30 wt %) is provided to the methanol reactor 210 by a line 219. A weak NaBr/water solution is also delivered to the methanol reactor 210 by a line 220.

In the methanol formation reactor, methyl bromide reacts with water in the presence of strong base (e.g., sodium hydroxide), and methanol is formed, along with possible byproducts such as formaldehyde or formic acid. A liquid stream containing methanol, by-products, aqueous sodium bromide, and aqueous sodium hydroxide is carried away from the reactor via a line 221, to a stripper 222. A portion of the bottom liquid from the reactor/absorber 210 is circulated via a line 223 through a cooler 224 to control temperature in the reactor/absorber 210.

The stripper 222 is equipped with a reboiler 225 and, optionally, a partial reflux. Aqueous sodium bromide and sodium hydroxide are removed with most of the water as the “bottoms” stream of the stripper. The vapor exiting the top of the stripper is carried by a line 226 to another distillation unit 227 equipped with a reboiler 228 and a condenser 229. In the distillation unit 227, by-products are separated from methanol, and the methanol is removed from the distillation unit 227 via a line 230, through a cooler 231, to a storage tank 232. The vapor from the distillation unit 227 (which may contain by-products) is carried via a line 233 through the condenser 229 and then through a line 234 to a by-product storage tank 235. Optionally, depending on the particular by-products produced and their boiling points, methanol may be taken as a distillate while by-products are recovered as bottoms.

The effluent stream removed from the distillation unit 222 and reboiler 225 contains water and aqueous sodium bromide and sodium hydroxide. This is carried away from the distillation unit via a line 236 and cooled by passing through a cooler 237 before being delivered to a sodium bromide holding tank 238. It is desirable to lower the pH of this salt solution. This is accomplished by metering the delivery of aqueous HBr from the hydrogen bromide holding tank 218 via a line 239 to a pH control device 240 coupled to the sodium bromide holding tank 238.

With the pH of the sodium bromide in the holding tank 238 brought to the desired level (e.g., slightly acidic), aqueous sodium bromide is removed from the tank and carried via a line 241 through a filter 242, and delivered to an electrolytic cell 243, having an anode 244 and a cathode 245. The filter is provided to protect the membranes in the electrolytic cells. Preferably, a series of electrolytic cells, rather than a single cell, is used as an electrolyzer.

Aqueous sodium bromide is electrolyzed in the electrolytic cell(s), with bromide ion being oxidized at the anode (2Br⁻→Br₂+2e⁻) and water being reduced at the cathode (2H₂O+2e⁻→H₂+2OH⁻). This results in the formation of sodium hydroxide, which is carried away from the electrolyzer as an aqueous solution via line 246 to a holding tank 247. The sodium hydroxide solution is then routed to the methanol reactor 210 via a line 219.

Molecular bromine is removed from the electrolyzer via a line 248 to a compressor 249, and then to a dryer 250. The bromine is returned to the bromination reactor 203 by passing it through a heat exchanger 205 and, if necessary, a heater 251. Molecular bromine that is dissolved in the anolyte is also removed from the electrolytic cell(s) 243 by carrying the anolyte from the cell(s) via a line 252 to a stripper 253, where bromine is removed by stripping with natural gas (supplied via a line 254) or by other means. The molecular bromine is carried by a line 255 to the compressor 249, dryer 250, etc., before being returned to the bromination reactor as described above.

Hydrogen generated in the electrolyzer is removed by a line 256, compressed in a compressor 257 and, optionally, routed to a power generation unit 258. Residual methane or other inert gasses can be removed from the methanol formation reactor via a line 259. The methane or natural gas can be routed to the power generation unit 258 to augment power generation. Additional natural gas or methane can be supplied to the unit via a line 260 if needed.

In a laboratory implementation of elements of the process depicted in FIG. 3, methane is reacted with gaseous bromine at about 450° C. in a glass tube bromination reactor, with a space time is a 60 seconds. The products are methyl bromide, HBr, and dibromomethane with a methane conversion of 75%. In the methanol formation reactor, the methyl bromide, HBr, and dibromomethane, react with water in the presence of sodium hydroxide to form methanol and formaldehyde (from the dibromomethane). It is further demonstrated that the formaldehyde is disproportionated to methanol and formic acid. Hence, overall, the products are methanol and formic acid.

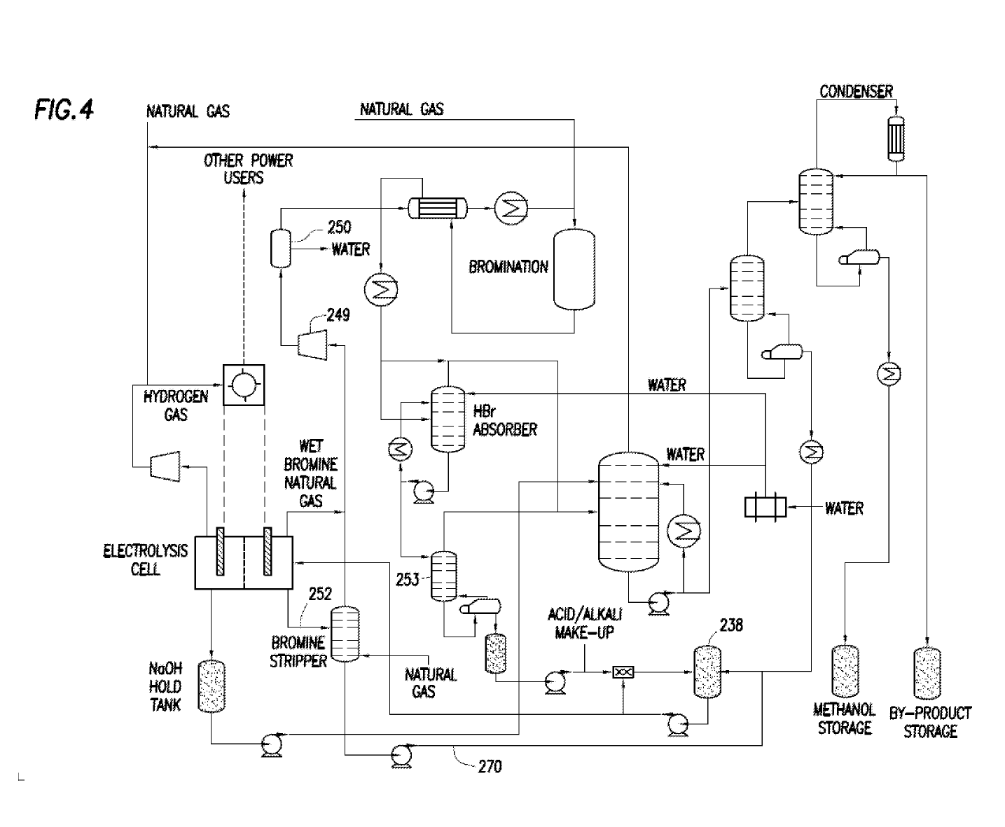

The process shown in FIG. 3 employs membrane-type electrolytic cells, rather than diaphragm-type cells. In a membrane cell, sodium ions with only a small amount of water flow to the cathode compartment. In contrast, in a diaphragm-type cell, both sodium ions and water proceed into the cathode compartment. In an alternate embodiment of the invention shown in FIG. 4, diaphragm cells are used, resulting in continuous depletion of the anolyte with respect to NaBr. To replenish the NaBr, depleted anolyte is taken through a line 252 to a bromine stripper 253 where bromine is removed and carried to a compressor 249 and then a dryer 250. NaBr solution from the stripper 253 is carried by a line 270 to the NaBr holding tank 238, where it combines with a richer NaBr solution. Other features of the process are similar to those in FIG. 3.

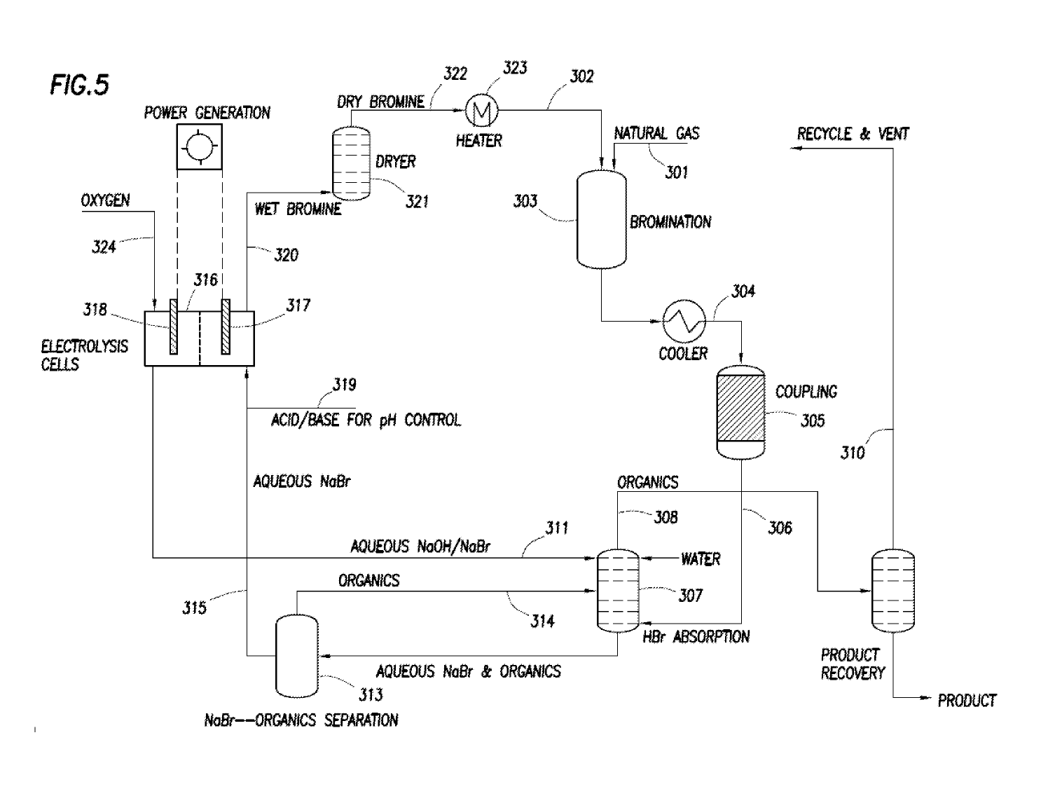

In another aspect of the invention, molecular halogen is recovered by electrolysis using a non-hydrogen producing cathode, e.g., an oxygen depolarized cathode, which significantly reduces the power consumption by producing water instead of hydrogen. FIG. 5 depicts one embodiment of this aspect of the invention, in this case involving the production of higher hydrocarbons. The flow diagram is similar to that shown in FIG. 1, with the differences noted below.

Bromine and natural gas, methane, or another light hydrocarbon are caused to react in a bromination reactor 303, and followed by a coupling reactor 305. The organics and HBr are separated in an HBr absorption unit 307. Aqueous sodium bromide is carried via line 315 to an electrolytic cell 316 equipped with an anode 317, oxygen depolarized cathode 318, and an oxygen inlet manifold or line 324. Optionally, additional water or electrolyte or pH control chemicals are carried into the cell via a line 319.

Molecular bromine is generated at the anode (2Br⁻→Br₂+2e⁻), and the wet bromine is carried via a line 320 to a dryer 321, through a heater 323, and then routed back to the bromination reactor 303. At the cathode, oxygen is electrolytically reduced in the presence of water (2H₂O+2e⁻→H₂+2OH⁻) (½O+H₂O+2e⁻→2OH⁻), and hydroxyl ions are carried away as aqueous sodium hydroxide, via line 311, to the HBr absorption unit 307.

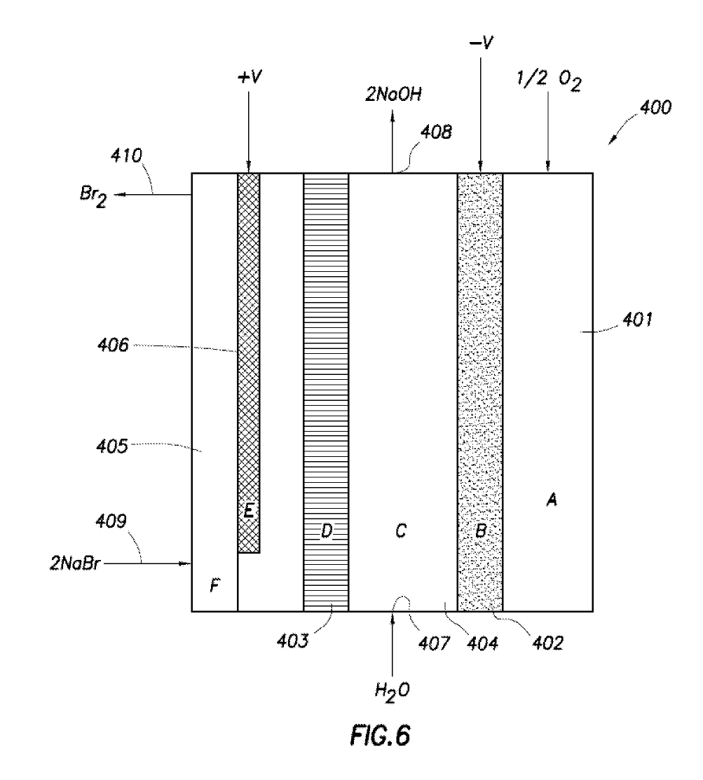

The invention also provides an improved electrolytic cell for converting halides into molecular halogen, one embodiment of which is shown in FIG. 6. The cell 400 includes a gas supply manifold 401, through which oxygen gas, air, or oxygen-enriched air can be introduced; a gas diffusion cathode 402, which is permeable to oxygen (or an oxygen-containing gas); a cation exchange membrane 403; a cathode electrolyte chamber 404 disposed between the cation exchange membrane and the gas diffusion cathode; an anode electrolyte chamber 405; and an anode 406, extending into the anode electrolyte chamber. When operating under basic (alkaline) conditions, water is introduced into the cathode electrolyte chamber through a port 407, and aqueous sodium hydroxide is removed from the chamber via another port 408. Similarly, aqueous sodium bromide is introduced into the anode electrolyte chamber through a port 409, and molecular bromine is carried away from the anode electrolyte chamber via a line 410. The anode and cathode can be connected to an electrical power supply (not shown), which may include equipment for converting AC to DC current (e.g., mechanical rectifier, motor-generator set, semiconductor rectifier, synchronous converter, etc.) and other components.

In operation, water is introduced into the cathode electrolyte chamber through the water inlet port 407, and aqueous sodium bromide is introduced into the anode electrolyte chamber 405 through port 409. Oxygen flow through the gas supply manifold 401 is commenced and the power to the cell is turned on. Sodium bromide is reduced at the anode, bromine gas is evolved and carried away by line 410, and sodium ions are carried through the cation exchange membrane into the cathode electrolyte chamber. At the cathode, oxygen is electrolytically reduced to hydroxyl ion in the presence of water. Aqueous sodium hydroxide exits the cathode electrolyte chamber through port 408.

The electrolytic cell described herein can be used in conjunction with various processes, including the embodiments presented above. It is particularly advantageous when power consumption is an issue, and where it is desirable not to form hydrogen (e.g., where the risk of fire warrants extra precautions, such as on an offshore drilling rig).

Although the invention can be used in a variety of industrial settings, particular value is realized where a continuous process as described herein for making, e.g., higher hydrocarbons or methanol, is carried out at an offshore oil rig or drilling platform, or at a facility located onshore in a remote location. Part of the utility lies in the conversion of a difficult to transport material (e.g., natural gas) into a more easily transported liquid material, such as higher hydrocarbons or methanol. Another utility resides in the use of the production facility's existing electrical generation capacity, such as an electrical generator or other power supply.

According to one embodiment of this aspect of the invention, an improved production facility where oil or gas is pumped from a well and thereby extracted from the earth is provided, the facility having an electrical generator or other electrical power supply, the improvement comprising: (a) forming alkyl halides by reacting molecular halogen with oil or gas pumped from the well, under process conditions sufficient to form alkyl halides and hydrogen halide; optionally with substantially complete consumption of the molecular halogen; (b) forming higher hydrocarbons and hydrogen halide by contacting the alkyl halides with a first catalyst under process conditions sufficient to form higher hydrocarbons and hydrogen halide; (c) separating the higher hydrocarbons from hydrogen halide; and (d) converting the hydrogen halide into hydrogen and molecular halogen electrolytically, using electricity provided by the electrical generator or electrical power supply, thereby allowing the halogen to be reused.

In another embodiment, an improved production facility where oil or gas is pumped from a well and thereby extracted from the earth is provided, the facility having an electrical generator or other electrical power supply, the improvement comprising: (a) forming alkyl halides by reacting molecular halogen with a hydrocarbon feedstock under process conditions sufficient to form alkyl halides and hydrogen halide, optionally with substantially complete consumption of the molecular halogen; (b) forming methanol and alkaline halide by contacting the alkyl halides with aqueous alkali under process conditions sufficient to form methanol and alkaline halide; (c) separating the methanol from the alkaline halide; (d) converting the alkaline halide into hydrogen, molecular halogen, and aqueous alkali electrolytically, using electricity provided by the electrical generator or electrical power supply, thereby allowing the halogen and the alkali to be reused.

In another aspect of the invention, the general approach described above, including the steps of halogenation, product formation, product separation, and electrolytic regeneration of halogen is used to make alkyl amines. Thus, in one embodiment, natural gas, methane, or another aliphatic hydrocarbon feedstock is converted into alkyl amines via intermediate alkyl bromides. The feedstock and gaseous bromine are carried by separate lines into a bromination reactor and allowed to react. The bromination products (e.g., methyl bromide and HBr), and possibly unreacted hydrocarbons, are carried by a line through a heat exchanger, which lowers their temperature. The alkyl bromides are then carried by a line to an amination reactor. Ammonia or aqueous ammonia is also provided to the amination reactor by a separate line. The alkyl bromide and ammonia are allowed to react under process conditions sufficient to form alkyl amines (e.g., RN₂) and sodium bromide, which are then separated in a manner analogous to that described above with respect to the production of methanol. Aqueous sodium bromide is carried by a line to an electrolytic cell or cells, where it is converted into hydrogen and molecular bromine electrolytically, thereby allowing the bromine to be reused in the next cycle.

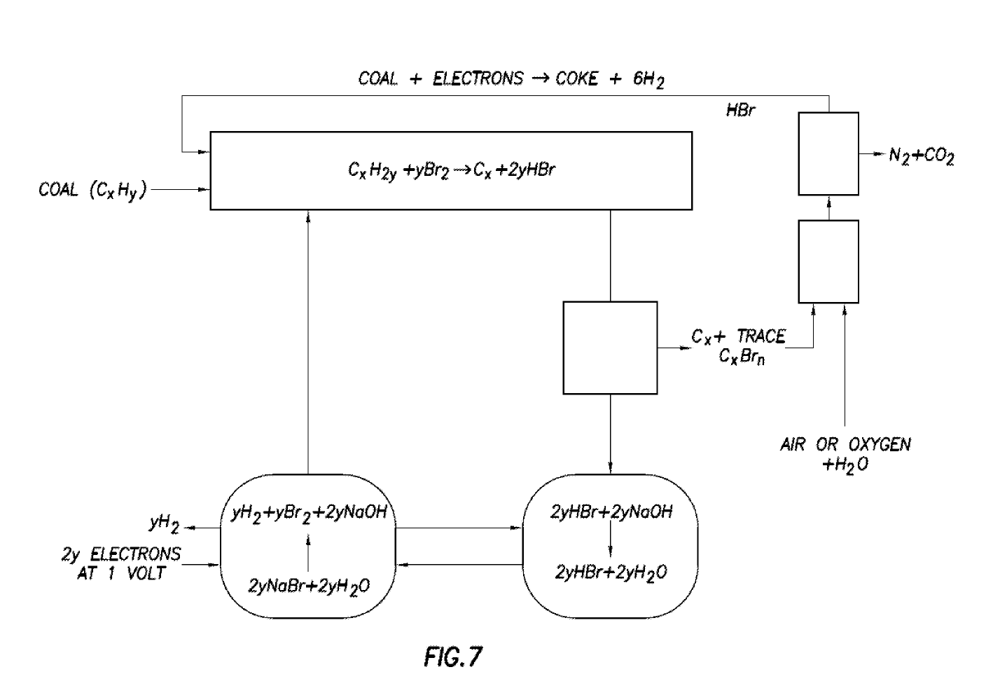

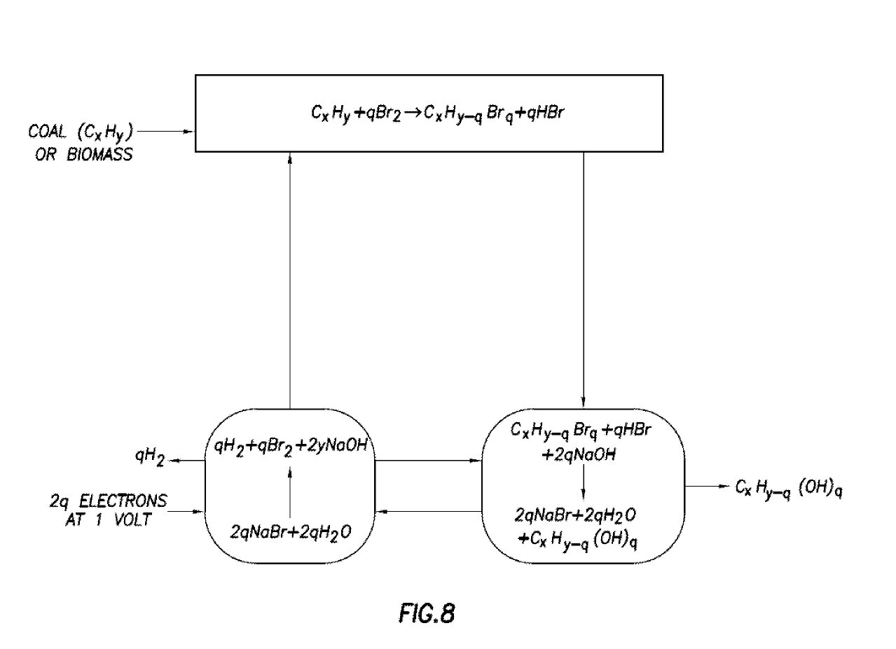

Referring now to FIGS. 7 and 8, two other aspects of the invention are presented, in which coal is converted to higher value coke, or coal or biomass is converted into higher value polyols (poly-alcohols), and the halogen used in the process is regenerated electrolytically. In the embodiments shown in FIG. 7, crushed coal is allowed to react with molecular bromine at elevated temperature, forming coke, HBr, and brominated coal intermediates (“CₓBrₙ”). The brominated coal intermediates are converted into coke by allowing them to contact a catalyst, thereby forming additional hydrogen bromide. The coke and hydrogen bromide are then separated, and the hydrogen bromide is then carried by a line to an electrolytic cell or cells, similar to that described above, thereby allowing molecular bromine to be regenerated and reused.

FIG. 8 depicts a similar process in which coal or biomass-derived hydrocarbons are brominated, thereby forming alkyl bromines or alkyl bromides and HBr, which are then processed in a manner analogous to that described above, e.g., the alkyl bromides and HBr are at least partially separated and the alkyl bromides are allowed to react with alkali, (e.g., sodium hydroxide), thereby forming sodium bromide, water, and poly-alcohols (“CₓH_{y-q}(OH)_{q}”). The poly-alcohols are separated from sodium bromide, and the aqueous sodium bromide is carried by a line to an electrolytic cell or cells, where molecular bromine is regenerated and subsequently separated and reused.

Referring to FIGS. 12-15, other aspects of the invention are presented using photoelectrocatalytic cells. Referring to FIG. 12, an alternate embodiment for converting natural gas, methane, or other hydrocarbon feedstocks into higher hydrocarbons, such as fuel grade hydrocarbons and aromatic compounds, is depicted. In this embodiment, HBr oxidation takes place in a photoelectrocatalytic cell. Natural gas may be used as a feed for this process and passed through a bromination reactor and coupling reactor as described in detail above. Products from the coupling reactor (e.g., higher hydrocarbons and HBr) may be carried to an HBr separation unit, where hydrocarbon products are separated from HBr. The HBr may then be carried to the photoelectrocatalytic cell. In an embodiment, an aqueous HBr electrolyte may be electrolyzed using a solar powered photoelectrocatalytic cell as described in more detail above, forming molecular bromine and hydrogen. The resulting electrolyte may have a decreased HBr concentration. The electrolyte may be further electrolyzed to fully remove the HBr or may be recirculated to the HBr absorption unit in a cyclic process. The hydrogen may be separated from the Br₂ for use within the process or exported for use or sale. The wet bromine may be carried to a dryer, where it may be dried before being reintroduced into the bromination reactor.

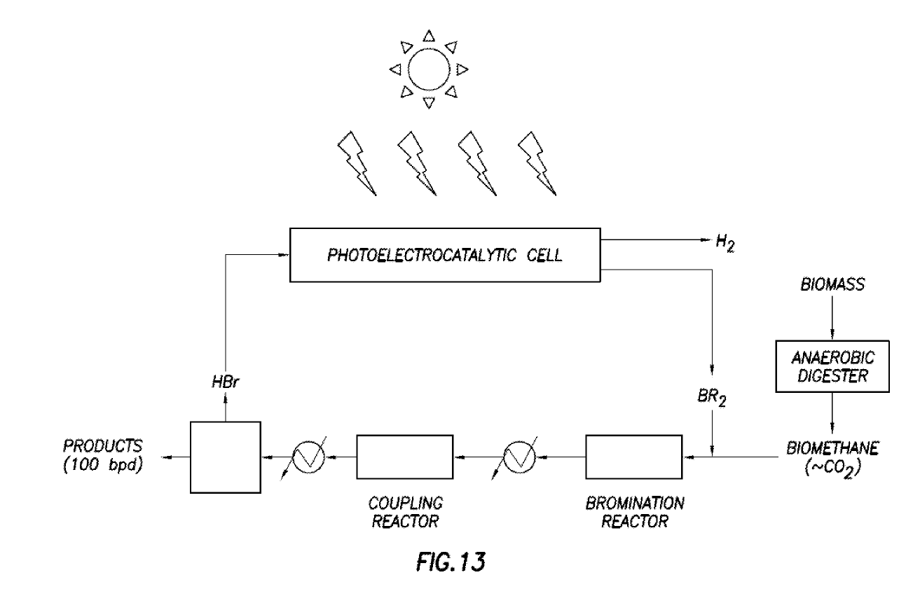

Referring to FIG. 13 still another embodiment for converting natural gas, methane, or other hydrocarbon feedstocks into higher hydrocarbons, such as fuel grade hydrocarbons and aromatic compounds, is depicted. In this embodiment, HBr oxidation takes place in a photoelectrocatalytic cell. In this embodiment, biomass may be fed to a conventional anaerobic digester to produce methane and possibly carbon dioxide for use as a feedstock within the process. The methane may be passed through a bromination reactor and coupling reactor as described in detail above. In this process some carbon dioxide may be present due to the source of the hydrocarbons used in the process. Products from the coupling reactor (e.g., higher hydrocarbons, HBr, and any unreacted gases) may be carried to an HBr separation unit, where hydrocarbon products may be separated from HBr. Any carbon dioxide present may either be separated or passed with the HBr to the photoelectrocatalytic cell. The HBr and any carbon dioxide present may be electrolyzed using a solar powered photoelectrocatalytic cell as described in more detail above. If carbon dioxide is present in the feed to the photoelectrocatalytic cell, various organic products may be formed along with molecular bromine and water. For example, carbon dioxide may be converted into methanol or formic acid in the photoelectrocatalytic cell along with molecular bromine according to the equations presented above. If the carbon dioxide is separated prior to the HBr stream passing to the photoelectrocatalytic cell, then molecular hydrogen and molecular bromine may be produced rather than any organic products. The resulting electrolyte may have a decreased HBr concentration. The electrolyte may be further electrolyzed to fully remove the HBr and any carbon dioxide present or may be recirculated to the HBr absorption unit in a cyclic process. Any organic products or hydrogen formed in the photoelectrocatalytic cell may be separated from the Br₂. The wet bromine may be carried to a dryer, where it may be dried before being reintroduced into the bromination reactor.

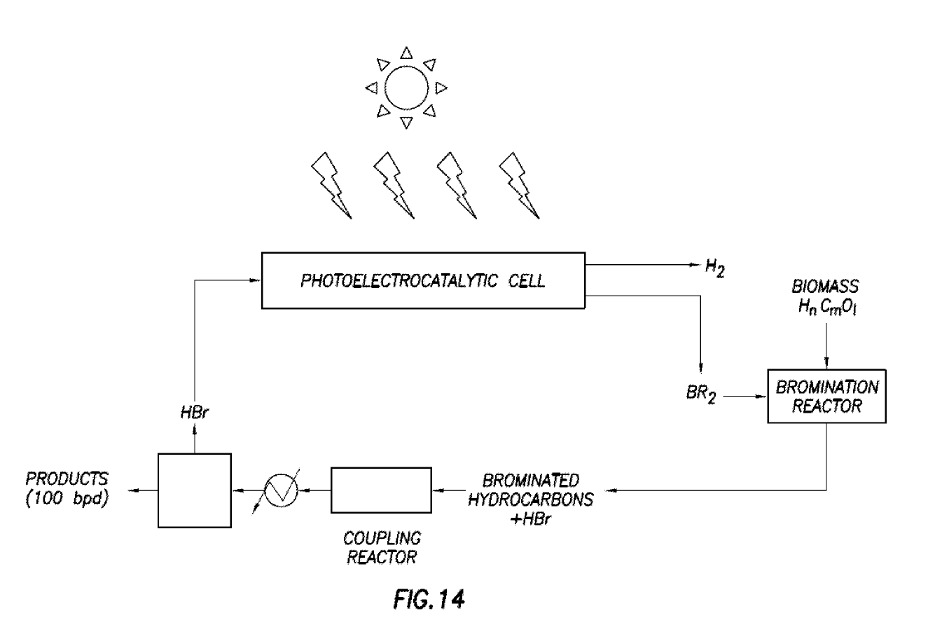

Referring to FIG. 14 yet another alternate embodiment for converting natural gas, methane, or other hydrocarbon feedstocks into higher hydrocarbons, such as fuel grade hydrocarbons and aromatic compounds, is depicted. In this embodiment, HBr oxidation takes place in a photoelectrocatalytic cell. In this embodiment, biomass may be directly reacted and decomposed in a halogen source such as bromine using a reactive distillation process. The reactive distillation process may replace the bromination reactor used with gaseous hydrocarbon sources. The products resulting from the reactive distillation process may include brominated poly and mono saccharides, water, and HBr. The products from the reactive distillation process may then be fed into a coupling reactor to form higher hydrocarbons and HBr. Products from the coupling reactor (e.g., higher hydrocarbons, HBr, and any unreacted gases) may be carried to an HBr absorption unit, where hydrocarbon products are separated from HBr. The aqueous HBr may be electrolyzed using a solar powered photoelectrocatalytic cell as described in more detail herein. Molecular hydrogen and molecular bromine may be produced in the photoelectrocatalytic cell. The resulting electrolyte may have a decreased HBr concentration. The electrolyte may be further electrolyzed to fully remove the HBr or may be recirculated to the HBr absorption unit in a cyclic process. Any hydrogen formed in the photoelectrocatalytic cell may be separated from the Br₂. The wet bromine may be carried to a dryer, where it may be dried before being reintroduced into the bromination reactor.

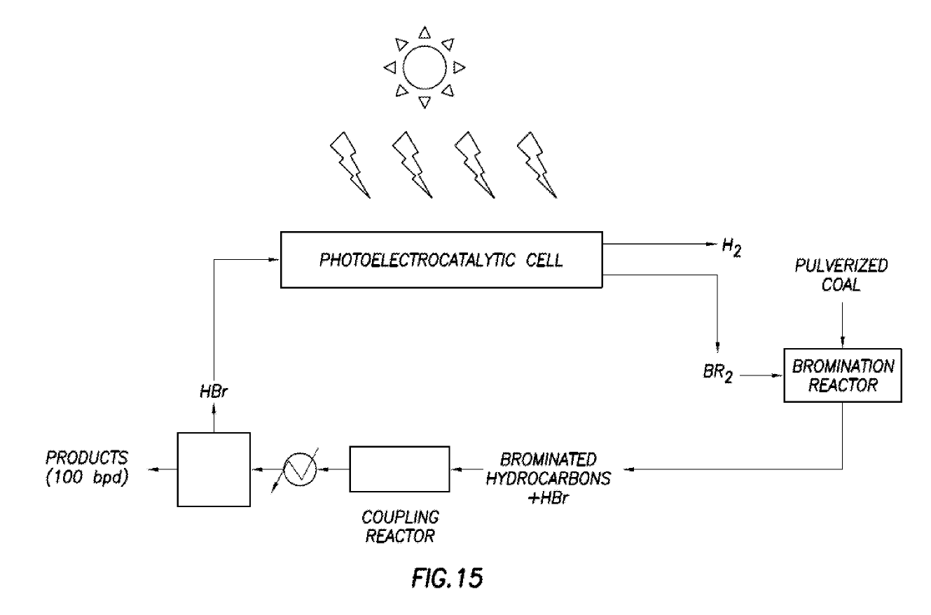

Referring to FIG. 15 yet another alternate embodiment for converting natural gas, methane, or other hydrocarbon feedstocks into higher hydrocarbons, such as fuel grade hydrocarbons and aromatic compounds, is depicted. In this embodiment, HBr oxidation takes place in a photoelectrocatalytic cell. In this embodiment, coal, which may be pulverized to form a powder, may be directly reacted and decomposed in a halogen source such as bromine using a reactive distillation process. The reactive distillation process may replace the bromination reactor used with gaseous hydrocarbon sources. The products resulting from the reactive distillation process may include brominated, liquefied fragments of the heterogeneous coal solids and HBr. The products from the reactive distillation process may then be fed into a coupling reactor for form higher hydrocarbons and HBr. Products from the coupling reactor (e.g., higher hydrocarbons, HBr, and any unreacted gases) may be carried to an HBr absorption unit, where hydrocarbon products are separated from HBr. The aqueous HBr may be electrolyzed using a solar powered photoelectrocatalytic cell as described in more detail herein. Molecular hydrogen and molecular bromine may be produced in the photoelectrocatalytic cell. The resulting electrolyte may have a decreased HBr concentration. The electrolyte may be further electrolyzed to fully remove the HBr and any carbon dioxide present or may be recirculated to the HBr absorption unit in a cyclic process. Any hydrogen formed in the photoelectrocatalytic cell may be separated from the Br₂. The wet bromine may be carried to a dryer, where it may be dried before being reintroduced into the bromination reactor.

Alternate embodiments have been described above with reference to FIGS. 12-15, wherein a photoelectrocatalytic cell is used in place of an electrolytic cell, thereby enabling the practitioner to form molecular halogen via photoelectrocatalysis. It is further understood that the photoelectrocatalytic cell can be substituted for the electrolytic cell in any of the other embodiments described above, for example the embodiments described with reference to FIGS. 1-5, 7 and 8, likewise enabling the practitioner to form molecular halogen via photoelectrocatalysis in these embodiments rather than by simple electrolysis.

The following nonlimiting examples illustrate various embodiments or features of the invention, including methane bromination, C—C coupling to form higher hydrocarbons, e.g., light olefins and aromatics (benzene, toluene, xylenes (“BTX”)), hydrolysis of methyl bromide to methanol, hydrolysis of dibromomethane to methanol and formaldehyde, and subsequent disproportionation to formic acid.

### Example 1

### Bromination of Methane

Methane (11 sccm, 1.0 atm) was combined with nitrogen (15 sccm, 1.0 atm) at room temperature via a mixing tee and passed through an 18° C. bubbler full of bromine. The CH₄/N₂/Br₂ mixture was passed into a preheated glass tube (inside diameter 2.29 cm, length, 30.48 cm, filled with glass beads) at 500° C., where bromination of methane took place with a residence time of 60 seconds, producing primarily bromomethane, dibromomethane and HBr:

CH₄+Br₂→CH₃Br+CH₂Br₂+HBr

As products left the reactor, they were collected by a series of traps containing 4M NaOH, which neutralized the HBr and hexadecane (containing octadecane as an internal standard) to dissolve as much of the hydrocarbon products as possible. Volatile components like methane were collected in a gas bag after the HBr/hydrocarbon traps.

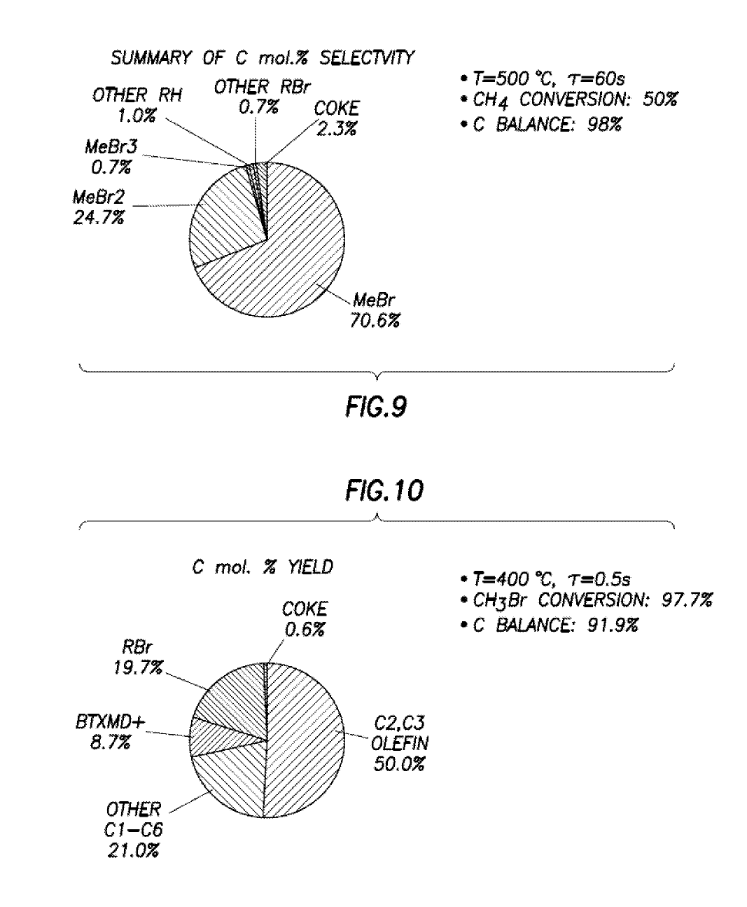

After the bromination reaction, the coke or carbonaceous deposits were burned off in a flow of heated air (5 sccm) at 500° C. for 4 hours, and the CO₂ was captured with a saturated barium hydroxide solution as barium carbonate. All products were quantified by GC. The amount of coke was determined based on the CO₂ evolution from decoking. The results are summarized in FIG. 9.

### Example 2

### CH₃Br Coupling to Light Olefins

2.27 g of a 5% Mg-doped ZSM-5 (CBV8014) zeolite was loaded in a tubular quartz reactor (1.0 cm ID), which was preheated to 400° C. before the reaction. CH₃Br, diluted by N₂, was pumped into the reactor at a flow rate of 24 μl/min for CH₃Br, controlled by a micro liquid pump, and 93.3 ml/min for N₂. The CH₃Br coupling reaction took place over the catalyst bed with a residence time of 0.5 sec and a CH₃Br partial pressure of 0.1 based on this flow rate setting.

After one hour of reaction, the products left the reactor and were collected by a series of traps containing 4M NaOH, which neutralized the HBr and hexadecane (containing octadecane as an internal standard) to dissolve as much of the hydrocarbon products as possible. Volatile components like methane and light olefins were collected in a gas bag after the HBr/hydrocarbon traps.

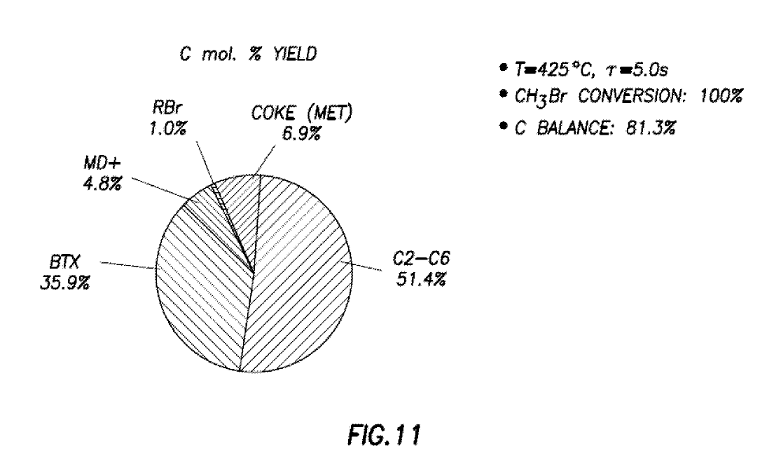

After the coupling reaction, the coke or carbonaceous deposits were burned off in a flow of heated air (5 sccm) at 500° C. for 4 hours, and the CO₂ was captured with a saturated barium hydroxide solution as barium carbonate. All products were quantified by GC. The amount of coke was determined based on the CO₂ evolution from decoking. The results are summarized in FIG. 10.

Even at such a short residence time, CH₃Br conversion reached 97.7%. Among the coupling products, C₃H₆ and C₂H₄ are the major products, and the sum of them contributed to 50% of carbon recovery. BTX, other hydrocarbons, bromohydrocarbons and a tiny amount of coke made up the balance of the converted carbon.

### Example 3

### CH₃Br Coupling to BTX

Pellets of Mn ion exchanged ZSM-5 zeolite (CBV3024, 6 cm in length) were loaded in a tubular quartz reactor (ID, 1.0 cm), which was preheated to 425° C. before the reaction. CH₃Br, diluted by N₂, was pumped into the reactor at a flow rate of 18 μl/min for CH₃Br, controlled by a micro liquid pump, and 7.8 ml/min for N₂. The CH₃Br coupling reaction took place over the catalyst bed with a residence time of 5.0 sec and a CH₃Br partial pressure of 0.5 based on this flow rate setting.

After one hour of reaction, the products left the reactor and were collected by a series of traps containing 4M NaOH, which neutralized the HBr and hexadecane (containing octadecane as an internal standard) to dissolve as much of the hydrocarbon products as possible. Volatile components like methane and light olefins were collected in a gas bag after the HBr/hydrocarbon traps.

After the coupling reaction, the coke or carbonaceous deposits were burned off in a flow of heated air (5 sccm) at 500° C. for 4 hours, and the CO₂ was captured with a saturated barium hydroxide solution as barium carbonate. All products were quantified by GC. The amount of coke was determined based on the CO₂ evolution from decoking. The results are summarized in FIG. 8.

With this BTX maximum operation mode, CH₃Br can be converted completely. BTX yield reached 35.9%. Other hydrocarbons, aromatics, bromohydrocarbons, and coke contributed to the carbon recovery of 51.4%, 4.8%, 1.0%, and 6.9% respectively. Propane is a major components of the “other hydrocarbons,” and can be sent back for reproportionation followed by further coupling to boost the overall BTX yield even higher.

### Example 4

### Caustic Hydrolysis of Bromomethane to Methanol

CH₃Br+NaOH→CH₃OH+NaBr

In a 30 ml stainless steel VCR reactor equipped with a stir bar, 13.2 g 1M sodium hydroxide aqueous solution (13.2 mmol) and 1.3 g bromomethane (12.6 mmol) were added in sequence. The reactor was gently purged with nitrogen to remove the upper air before closing the cap. The closed reactor was placed in an aluminum heating block preheated to 150° C. and the reaction started simultaneously. The reaction was run for 2 hours at this temperature with stirring.

After stopping the reaction, the reactor was placed in an ice-water bath for a start time to cool the products inside. After opening the reactor, the reaction liquid was transferred to a vessel and diluted by cold water. The vessel was connected with a gas bag used to collect the un-reacted bromomethane, if any. The reaction liquid was weighed and the product concentrations were analyzed with a GC-FID, in which an aqueous injection applicable capillary column was installed.

The gas product analysis shows that there was no bromomethane remaining, indicating that bromomethane was converted completely. Based on the concentration measurements for the liquid product, the methanol yield including tiny amount of dimethyl ether, was calculated to be 96%.

### Example 5

### Caustic Hydrolysis of Dibromomethane to Formaldehyde Followed by Disproportionation to Methanol and Formic Acid

CH₂Br₂+2NaOH→HCHO+2NaBr+H₂O

HCHO+½H₂O→½CH₃OH+½HCOOH

Caustic hydrolysis of dibromomethane was carried out according to the same procedure as in Example 5, with the exception that a high NaOH/CH₂Br₂ ratio (2.26) was employed. After collecting the reaction liquid, a sufficient quantity of concentrated hydrogen chloride solution was added to neutralize the extra sodium hydroxide and acidify sodium formate. Methanol and formic acid were observed to be the only products, indicating that hydrolysis to methanol and formaldehyde was followed by complete disproportionation of formaldehyde to (additional) methanol and formic acid. The GC analysis shows that the conversion of dibromomethane reached 99.9%; while the yields of methanol and formic acid reached 48.5% and 47.4% respectively.

Examples 4 and 5 demonstrate that bromomethane can be completely hydrolyzed to methanol, and dibromomethane can be completely hydrolyzed to methanol and formic acid, under mild caustic conditions. The results are summarized in Table 1.

TABLE 1
Caustic Hydrolysis of CH₃Br and CH₂Br₂ and
Subsequent Disproportionation of HCHO

Starting from

| | CH₂Br | CH₂Br₂ |
|---|---|---|
| NaOH/CH₃Br or CH₂Br₂ | 1.05 | 2.17 |
| Temperature (° C.) | 150 | 150 |
| Reaction Time (hr) | 2 | 2 |
| Conversion (%) | 100.0 | 99.9 |
| CH₃OH Yield (%) | 96.0 | 48.5 |
| HCOOH Yield (%) | | 47.4 |

The invention has been described with reference to various representative and preferred embodiments, but is not limited thereto. Other modifications and equivalent arrangements, apparent to a skilled person upon consideration of this disclosure, are also included within the scope of the invention. As one example, molecular bromine may also be removed from the electrolytic cell(s) using a concurrent extraction technique, wherein an inert organic solvent, such as chloroform, carbon tetrachloride, ether, etc. is used. The solvent may be introduced on one side of a cell; bromine partitions between the aqueous and organic phases; and bromine-laden solvent may be withdrawn from another side of the cell. Bromine may then be separated from the solvent by distillation or another suitable technique and then returned to the system for reuse. Partitioning is favored by bromine's significantly enhanced solubility in solvents such as chloroform and carbon tetrachloride, as compared to water. Extraction in this way serves a dual purpose: it separates Br₂ from other forms of bromine that may be present (e.g., Br⁻, OBr⁻, which are insoluble in the organic phase); and it allows bromine to be concentrated and easily separated from the organic phase (e.g., by distillation). An optimal pH for extraction (as well as for separation of bromine by heating bromine-containing aqueous solutions in a gas flow) is pH 3.5—the pH at which the concentration of molecular bromine (Br₂) is at its highest, as compared to other bromine species.

As another example of modifications to the process disclosed herein, various pumps, valves, heaters, coolers, heat exchangers, control units, power supplies, and equipment in addition or in the alternative to that shown in the figures can be employed to optimize the processes. In addition, other features and embodiments may be utilized in the practice of the present invention. The invention is limited only by the accompanying claims and their equivalents.

Both in this written description and in the claims, when chemical substances are referred to in the plural, singular referents are also included, and vice versa, unless the context clearly dictates otherwise. For example, “alkyl halides” includes one or more alkyl halides, which can be the same (e.g., 100% methyl bromide) or different (e.g., methyl bromide and dibromomethane); “higher hydrocarbons” includes one or more higher hydrocarbons, which can be the same (e.g., 100% octane) or different (e.g., hexane, pentane, and octane).

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Moreover, the indefinite articles “a” or “an,” as used in the claims, are defined herein to mean one or more than one of the element that it introduces. Also, the terms in the claims have their plain, ordinary meaning unless otherwise explicitly and clearly defined by the patentee.

What is claimed is:

1. A method for converting a hydrocarbon feedstock into methanol, comprising:
(a) reacting a hydrocarbon feedstock with molecular halogen so as to form alkyl halides;
(b) contacting at least a portion of the alkyl halides with an alkali so as to form methanol and a halide salt; and
(c) photoelectrocatalytically converting at least a portion of the halide salt into molecular halogen.

2. The method of claim 1 wherein the hydrocarbon feedstock comprises natural gas.

3. The method of claim 1 wherein the hydrocarbon feedstock comprises methane.

4. The method of claim 1 further comprising repeating steps (a) through (c), wherein the molecular halogen in step (c) is used in repeated step (a).

5. The method of claim 1 further comprising separating the methanol from the halide salt prior to step (c).

6. The method of claim 1 wherein the halide salt is photoelectrocatalytically converted into molecular halogen by electrolyzing the halide salt in a photoelectrocatalytic cell.

7. The method of claim 1 wherein the hydrocarbon feedstock is derived from a biomass.

8. The method of claim 1 wherein the hydrocarbon feedstock comprises coal.

9. The method of claim 1 wherein the halide salt is photoelectrocatalytically converted into molecular halogen in a photoelectrocatalytic cell and wherein the method further comprises feeding carbon dioxide to the photoelectrocatalytic cell and photoelectrocatalytically converting the carbon dioxide into methanol, formic acid, or a mixture thereof.

10. The method of claim 1 wherein the halide salt is photoelectrocatalytically converted into molecular halogen in a photoelectrocatalytic cell that comprises an electrolyte, an anodic electrode, and a cathodic electrode.

11. The method of claim 10 wherein the anodic electrode, the cathodic electrode, or both have a flat sheet-like configuration.

12. The method of claim 10 wherein the anodic electrode, the cathodic electrode, or both have a tubular configuration.

13. The method of claim 10 wherein the anodic electrode, the cathodic electrode, or both have a particulate configuration.
